(19) United States
(12) Patent Application Publication
(10) Pub. No.: US 2015/0080945 A1
(43) Pub. Date: Mar. 19, 2015

(54) Partial Circumferential Stent with Non-Radial Apposition

(71) Applicant: W. L. Gore & Associates, Inc., Newark, DE (US)

(72) Inventor: Christopher S. Michalak, Elkton, MD (US)

(73) Assignee: W. L. Gore Associates, Inc., Newark, DE (US)

(21) Appl. No.: 14/030,535

(22) Filed: Sep. 18, 2013

(51) Int. Cl. A61B 17/00

(52) U.S. Cl. CPC A61B 17/0057; A61B 2017/00659
USPC 606/213

(57) ABSTRACT

A medical device for sealing and repairing defects in a body lumen wall includes a wire frame and a partially-circumferential stent body. In some embodiments, the endolumenal sealing devices provided herein are well-suited for use in the GI tract including the colon. That is the case because the partially-circumferential sealing devices are configured to be compliant with the peristaltic movements of the GI tract. This feature can enable the sealing devices to resiliently remain located in a desired position within the GI tract, such that the defect in the lumen wall is sealed by the presence of the sealing device.

### FIELD OF THE INVENTION

This document relates to implantable medical devices. For example, this document relates to endolumenal sealing devices for repairing defects in a body lumen wall, and to methods for treating defects using the endolumenal sealing devices.

### BACKGROUND

The need to remove lesions from the wall of the colon is common and growing worldwide. The likelihood of having polyps increases with age. Approximately half of the people over the age of 60 have at least one polyp and often more. Polyps are considered pre-cancerous, which means that while they are not cancer, if left untreated they may develop into cancer. Colon lesions are typically found during colon cancer screening tests, such as a colonoscopy or flexible sigmoidoscopy. Lesions of the colon can be in the form of polyps that protrude from the colon lining with a mushroom-like shape, or flat lesions that are flush on the colon wall.

Benign and early malignant lesions of the colon can usually be removed endoscopically using an electrocautery snare, hot snare, cold snare, or electrocautery knife devices. A saline-assisted polypectomy procedure is often used for the removal of large flat colon lesions. The procedure starts with injection of a solution into the submucosal space under the lesion, creating a safety cushion. The cushion lifts the lesion to facilitate its removal and minimizes mechanical or electrocautery damage to the deep layers of the GI tract wall.

When lesions become still larger and invasively encompass more than just the mucosal layers of the colon, a colectomy procedure is often performed whereby the full thickness of the colon wall tissue is removed along with the lesion. This procedure is typically performed using laparoscopic or open surgery techniques rather than endoscopically. Large resections of the colon are not typically performed endoscopically in part because tools and devices to adequately seal the resulting perforation in the colon wall are not available. Such tools and devices are challenging to develop in part because of the relatively hostile colon environment that includes peristaltic movements and fecal matter.

### SUMMARY

This document provides implantable medical devices. For example, this document provides partially-circumferential endolumenal sealing devices for sealing and repairing defects in a body lumen wall, and methods for treating lumen wall defects using the endolumenal sealing devices. Such defects can include, but are not limited to, vessel aneurysms and GI tract perforations. In some embodiments, the endolumenal sealing devices provided herein are well-suited for use in the GI tract including the colon. That is the case because the sealing devices can be configured to be partially-circumferential, whereby the device can be compliant with the peristaltic movements of the GI tract. This feature can enable the sealing devices to resiliently remain located in a desired position within the GI tract, such that the defect in the lumen wall is sealed by the presence of the sealing device.

In general, one aspect of this document features an implantable medical device for treating a portion of a body lumen wall. The device comprises a membrane frame; a membrane material disposed on at least a portion of the membrane frame; a first strut extending from the membrane frame; and a second strut extending from the membrane frame, wherein the first and second struts are configured to exert opposing non-orthogonal forces to the membrane frame to thereby press at least a portion of the membrane material into contact with the body lumen wall.

In various implementations, the portion of the body lumen wall being treated by the device may comprise a defect, and the membrane material of the device may be configured to overlay the defect. The first and second struts may optionally each comprise elongate members that are unitary with an elongate member of the membrane frame. The first and second struts may optionally each comprise elongate members that are joined to an elongate member of the membrane frame. The first and second struts may optionally each comprise elongate members that are joined to an elongate member of the membrane frame by one or more of a weld, an adhesive, and a sleeve. The first and second struts may extend from a perimeter of the membrane frame. The first and second struts may each comprise a discrete elongate member that is attached to the membrane frame at two or more points, and that forms a loop that is shaped as a generally elliptical segment when the device is in a fully expanded configuration. The first and second struts may have substantially identical shapes when the device is in a fully expanded configuration. In some embodiments, the membrane material may cover substantially the entire area defined by the membrane frame. In some embodiments, the device may assume a low-profile configuration when the device is maintained in a constraining environment, and the device may expand from the low-profile configuration and assumes an expanded configuration when the device is liberated from the constraining environment. The membrane frame may optionally be configured to contact an arcuate portion of the body lumen wall of about 120 to 180 degrees. The membrane frame may optionally be configured to contact an arcuate portion of the body lumen wall of about 60 to 120 degrees. In some embodiments, one or more of the first and second struts may include hinge points such that the one or more of the first and second struts are pivotable in relation to the membrane frame. The membrane material may be optionally configured to inhibit tissue ingrowth and endothelialization into the membrane material. At least a portion of the membrane material may be configured to be separated from the device and remain in a body when the device is removed from the body after treating the portion of the body lumen wall. The device may further comprise a bioabsorbable component that facilitates the separation of the at least a portion of the membrane material from the device. At least a portion of the membrane material that is configured to be separated from the device may be configured to promote tissue ingrowth or endothelialization into the membrane material, and in some embodiments the portion of the membrane material that is configured to promote tissue ingrowth or endothelialization into the membrane material may have an average porosity in the range of about 20 to 250 microns. In some embodiments, the portion of the membrane material that is configured to promote tissue ingrowth or endothelialization into the membrane material may have an average porosity in the range of about 100 to 200 microns. The portion of the membrane material that is configured to promote tissue ingrowth or endothelialization into the membrane material may include a plurality of openings that have an average diameter of about 0.25 to 2.0 millimeters. The portion of the membrane material that is configured to promote tissue ingrowth or endothelialization into the membrane material may include a coating of one or more growth factors. In some embodiments, the device further comprises an attachment feature configured for releasably coupling with a delivery device or a retrieval device. The membrane frame in some embodiments may further comprise one or more elongate elements extending between two locations on a perimeter of the membrane frame. The one or more elongate elements extending between two locations on the perimeter of the membrane frame may be non-linear. The device may optionally further comprise one or more radiopaque markers on the membrane frame, or one or more radiopaque markers on one or more of the first and second struts.

In various implementations, the portion of the body lumen wall may comprise a defect and the device may be configured to prevent protrusion of the membrane material into the defect. In some embodiments, the membrane frame has a length of about 0.5 to 15 centimeters. In some embodiments, the device may further comprise one or more tissue anchorage features on the membrane frame, on one or more of the first and second struts, or on both the membrane frame and one or more of the first and second struts. The struts may include atraumatic end portions. In some embodiments, at least portions of the first and second struts or the membrane frame may be coated with a coating that inhibits thrombus formation. Optionally, at least a portion of the membrane material may be modified by one or more chemical or physical processes to enhance particular properties of the materials.

In a second general aspect, this document features an implantable medical device for treating a portion of a body lumen wall, the device comprising: a membrane frame; a membrane material disposed on at least a portion of the membrane frame; a first strut extending from the membrane frame; and a second strut extending from the membrane frame, wherein the first and second struts are configured to press at least a portion of the membrane material into contact with the body lumen wall, and wherein the first and second struts each comprise an elongate member that extends from the membrane frame at two or more points and that forms a loop that is shaped as a generally elliptical segment when the device is in a fully expanded configuration.

In various implementations, the portion of the body lumen wall being treated by the device may comprise a defect, and the membrane material of the device may be configured to overlay the defect. The first and second struts may optionally each comprise elongate members that are unitary with an elongate member of the membrane frame. The first and second struts may optionally each comprise elongate members that are joined to an elongate member of the membrane frame. The first and second struts may optionally each comprise elongate members that are joined to an elongate member of the membrane frame by one or more of a weld, an adhesive, and a sleeve. The first and second struts may extend from a perimeter of the membrane frame. The first and second struts may each comprise a discrete elongate member that is attached to the membrane frame at two or more points. The first and second struts may have substantially identical shapes when the device is in a fully expanded configuration. In some embodiments, the membrane material may cover substantially the entire area defined by the membrane frame. In some embodiments, the device may assume a low-profile configuration when the device is maintained in a constraining environment, and the device may expand from the low-profile configuration and assumes an expanded configuration when the device is liberated from the constraining environment. The membrane frame may optionally be configured to contact an arcuate portion of the body lumen wall of about 120 to 180 degrees. The membrane frame may optionally be configured to contact an arcuate portion of the body lumen wall of about 60 to 120 degrees. In some embodiments, one or more of the first and second struts may include hinge points such that the one or more of the first and second struts are pivotable in relation to the membrane frame. The membrane material may be optionally configured to inhibit tissue ingrowth and endothelialization into the membrane material. At least a portion of the membrane material may be configured to be separated from the device and remain in a body when the device is removed from the body after treating the portion of the body lumen wall. The device may further comprise a bioabsorbable component that facilitates the separation of the at least a portion of the membrane material from the device. At least a portion of the membrane material that is configured to be separated from the device may be configured to promote tissue ingrowth or endothelialization into the membrane material, and in some embodiments the portion of the membrane material that is configured to promote tissue ingrowth or endothelialization into the membrane material may have an average porosity in the range of about 20 to 250 microns. In some embodiments, the portion of the membrane material that is configured to promote tissue ingrowth or endothelialization into the membrane material may have an average porosity in the range of about 100 to 200 microns. The portion of the membrane material that is configured to promote tissue ingrowth or endothelialization into the membrane material may include a plurality of openings that have an average diameter of about 0.25 to 2.0 millimeters. The portion of the membrane material that is configured to promote tissue ingrowth or endothelialization into the membrane material may include a coating of one or more growth factors. In some embodiments, the device further comprises an attachment feature configured for releasably coupling with a delivery device or a retrieval device. The membrane frame in some embodiments may further comprise one or more elongate elements extending between two locations on a perimeter of the membrane frame. The one or more elongate elements extending between two locations on the perimeter of the membrane frame may be non-linear. The device may optionally further comprise one or more radiopaque markers on the membrane frame, or one or more radiopaque markers on one or more of the first and second struts.

In various implementations, the portion of the body lumen wall may comprise a defect and the device may be configured to prevent protrusion of the membrane material into the defect. In some embodiments, the membrane frame has a length of about 0.5 to 15 centimeters. In some embodiments, the device may further comprise one or more tissue anchorage features on the membrane frame, on one or more of the first and second struts, or on both the membrane frame and one or more of the first and second struts. The struts may include atraumatic end portions. In some embodiments, at least portions of the first and second struts or the membrane frame may be coated with a coating that inhibits thrombus formation. Optionally, at least a portion of the membrane material may be modified by one or more chemical or physical processes to enhance particular properties of the materials.

In a third general aspect, this document features another implantable medical device for treating a portion of a body lumen wall. The device comprises: a membrane frame; a membrane material disposed on at least a portion of the membrane frame; a first strut extending from the membrane frame; and a second strut extending from the membrane frame, wherein the first and second struts are configured to press at least a portion of the membrane material into contact with the wall of the body lumen, and wherein the first and second struts each comprise an elongate member that is shaped as two arcuate segments and at least one wavy segment when the device is in a fully expanded configuration.

In various implementations, the portion of the body lumen wall being treated by the device may comprise a defect, and the membrane material of the device may be configured to overlay the defect. The first and second struts may optionally each comprise elongate members that are unitary with an elongate member of the membrane frame. The first and second struts may optionally each comprise elongate members that are joined to an elongate member of the membrane frame. The first and second struts may optionally each comprise elongate members that are joined to an elongate member of the membrane frame by one or more of a weld, an adhesive, and a sleeve. The first and second struts may extend from a perimeter of the membrane frame. The first and second struts may each comprise a discrete elongate member that is attached to the membrane frame at two or more points. The first and second struts may have substantially identical shapes when the device is in a fully expanded configuration. In some embodiments, the membrane material may cover substantially the entire area defined by the membrane frame. In some embodiments, the device may assume a low-profile configuration when the device is maintained in a constraining environment, and the device may expand from the low-profile configuration and assumes an expanded configuration when the device is liberated from the constraining environment. The membrane frame may optionally be configured to contact an arcuate portion of the body lumen wall of about 120 to 180 degrees. The membrane frame may optionally be configured to contact an arcuate portion of the body lumen wall of about 60 to 120 degrees. In some embodiments, one or more of the first and second struts may include hinge points such that the one or more of the first and second struts are pivotable in relation to the membrane frame. The membrane material may be optionally configured to inhibit tissue ingrowth and endothelialization into the membrane material. At least a portion of the membrane material may be configured to be separated from the device and remain in a body when the device is removed from the body after treating the portion of the body lumen wall. The device may further comprise a bioabsorbable component that facilitates the separation of the at least a portion of the membrane material from the device. At least a portion of the membrane material that is configured to be separated from the device may be configured to promote tissue ingrowth or endothelialization into the membrane material, and in some embodiments the portion of the membrane material that is configured to promote tissue ingrowth or endothelialization into the membrane material may have an average porosity in the range of about 20 to 250 microns. In some embodiments, the portion of the membrane material that is configured to promote tissue ingrowth or endothelialization into the membrane material may have an average porosity in the range of about 100 to 200 microns. The portion of the membrane material that is configured to promote tissue ingrowth or endothelialization into the membrane material may include a plurality of openings that have an average diameter of about 0.25 to 2.0 millimeters. The portion of the membrane material that is configured to promote tissue ingrowth or endothelialization into the membrane material may include a coating of one or more growth factors. In some embodiments, the device further comprises an attachment feature configured for releasably coupling with a delivery device or a retrieval device. The membrane frame in some embodiments may further comprise one or more elongate elements extending between two locations on a perimeter of the membrane frame. The one or more elongate elements extending between two locations on the perimeter of the membrane frame may be non-linear. The device may optionally further comprise one or more radiopaque markers on the membrane frame, or one or more radiopaque markers on one or more of the first and second struts.

In various implementations, the portion of the body lumen wall may comprise a defect and the device may be configured to prevent protrusion of the membrane material into the defect. In some embodiments, the membrane frame has a length of about 0.5 to 15 centimeters. In some embodiments, the device may further comprise one or more tissue anchorage features on the membrane frame, on one or more of the first and second struts, or on both the membrane frame and one or more of the first and second struts. The struts may include atraumatic end portions. In some embodiments, at least portions of the first and second struts or the membrane frame may be coated with a coating that inhibits thrombus formation. Optionally, at least a portion of the membrane material may be modified by one or more chemical or physical processes to enhance particular properties of the materials. In some embodiments, the at least one wavy segment of the struts is disposed between the two arcuate segments.

In a fourth general aspect, this document features a method of treating a defect in a body lumen wall. The method comprises: inserting an implantable medical device into the body lumen using a transcatheter technique, wherein the membrane material is configured to overlay the defect. The device comprises: a membrane frame; a membrane material disposed on at least a portion of the membrane frame; a first strut extending from the membrane frame; a second strut extending from the membrane frame, wherein the first and second struts are configured to exert opposing non-orthogonal forces to the membrane frame to thereby press at least a portion of the membrane material into contact with the wall of the body lumen; and one or more radiopaque markers attached to the device.

In various implementations, the method may further comprise visualizing the device during the inserting to confirm a positioning of the device as desired in relation to the defect. The method may further comprise detaching the device from a delivery device after confirming that the device has been positioned as desired in relation to the defect. In some embodiments, the defect may comprise an opening in the body lumen wall and the membrane material may be configured to fully overlay the opening. In some embodiments, the method may further comprise removing the device from the body lumen after the opening has been treated by the device. The method may optionally comprise, reconfiguring the device in a low-profile configuration prior to removal of the device. In some cases, the body lumen may comprise an intestine and the defect may comprise an opening in the wall of the intestine. In some cases, the intestine may be the colon. In some cases, the body lumen may be a blood vessel. In particular cases, the defect may be an aneurysm in the blood vessel. Optionally, the method may further comprise selecting the device based on a size of the defect. In some embodiments, the method may further comprise delivering a pharmacological agent to treat the defect.

In a fifth aspect, this document features a method of treating a defect in a body lumen wall. The method comprises: inserting a device into the body lumen using a transcatheter technique, wherein the membrane material is configured to overlay the defect. The device comprises: a membrane frame; a membrane material disposed on at least a portion of the membrane frame; a first strut extending from the membrane frame; a second strut extending from the membrane frame, wherein the first and second struts are configured to press at least a portion of the membrane material into contact with the body lumen wall, and wherein the first and second struts each comprise an elongate member that extends from the membrane frame at two or more points and that forms a loop that is shaped as a generally elliptical segment when the device is in a fully expanded configuration; and one or more radiopaque markers attached to the device.

In various implementations, the method may further comprise visualizing the device during the inserting to confirm a positioning of the device as desired in relation to the defect. The method may further comprise detaching the device from a delivery device after confirming that the device has been positioned as desired in relation to the defect. In some cases, the defect may comprise an opening in the body lumen wall and the membrane material may be configured to fully overlay the opening. In some embodiments, the method may further comprise removing the device from the body lumen after the opening has been treated by the device. The method may optionally comprise, reconfiguring the device in a low-profile configuration prior to removal of the device. In some cases, the body lumen may comprise an intestine and the defect may comprise an opening in the wall of the intestine. In some cases, the intestine may be the colon. In some cases, the body lumen may be a blood vessel. In particular cases, the defect may be an aneurysm in the blood vessel. Optionally, the method may further comprise selecting the device based on a size of the defect. In some embodiments, the method may further comprise delivering a pharmacological agent to treat the defect.

In a sixth general aspect, this document features an implantable medical device for treating a portion of a body lumen wall. The device comprises: a membrane frame; a membrane material disposed on at least a portion of the membrane frame; and two or more struts that each extend from the membrane frame and terminate at a free end, wherein the two or more struts are configured to exert non-orthogonal forces to the membrane frame to thereby press at least a portion of the membrane material into contact with the body lumen wall.

In various implementations, the device may comprise four struts. In some embodiments, the free ends may be configures to be atraumatic ends. Optionally, the two or more struts may each comprise elongate members that are joined to an elongate member of the membrane frame. The two or more struts may each comprise elongate members that are joined to an elongate member of the membrane frame by one or more of a weld, an adhesive, and a sleeve. The membrane frame may further comprise one or more elongate elements extending between two locations on a perimeter of the membrane frame. In some embodiments, the one or more elongate elements extending between two locations on the perimeter of the membrane frame may be non-linear. The device may further comprise one or more radiopaque markers on the membrane frame. The device may further comprise one or more radiopaque markers on one or more of the two or more struts.

Particular embodiments of the subject matter described in this specification can be implemented so as to realize one or more of the following advantages. In some embodiments, the partially-circumferential endolumenal sealing devices provided herein can be deployed into a body lumen of a patient using a minimally invasive transcatheter technique. The partially-circumferential endolumenal sealing devices can seal a defect in a body lumen wall so as to prevent the contents of the body lumen from exiting the lumen. The sealing function provided by the endolumenal sealing devices can promote healing of a body lumen wall defect by isolating the defect from the contents of the body lumen that may otherwise tend to inhibit the healing of the defect. In some embodiments, the partially-circumferential endolumenal sealing devices provided herein can be used treat aneurysms in blood vessels. The endolumenal sealing devices provide resilient fixation and consistent sealing of body lumen wall defects, even when positioned in body lumens that include anatomical movements—such as the peristaltic movements of the GI tract. Further, the endolumenal sealing devices are designed so as to not inhibit such movements. In some embodiments, the partially-circumferential endolumenal sealing devices provided herein can be deployed into a colon to seal a perforation related to a lesion resection. In such circumstances, the endolumenal sealing devices facilitate minimally invasive treatment of large colon lesions. In some embodiments, the partially-circumferential endolumenal sealing devices provided herein are repositionable and retrievable.

Other aspects, features, and advantages will be apparent from the description, the drawings, and the claims.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description, serve to explain the principles of the disclosure, wherein;

FIG. 1A is a perspective view of an example partially-circumferential endolumenal sealing device in accordance with some embodiments provided herein.

FIG. 1B is a perspective view of another example partially-circumferential endolumenal sealing device in accordance with some embodiments provided herein.

FIG. 2A illustrates the partially-circumferential endolumenal sealing device of FIG. 1A implanted in a body vessel to seal a perforation in the wall of the vessel.

FIG. 2B illustrates the partially-circumferential endolumenal sealing device of FIG. 1A implanted in a body vessel to seal an aneurysm in the wall of the vessel.

FIG. 3A is a perspective view of another example partially-circumferential endolumenal sealing device in accordance with some embodiments provided herein.

FIG. 3B is a perspective view of another example partially-circumferential endolumenal sealing device in accordance with some embodiments provided herein.

FIG. 4A is a perspective view of another example partially-circumferential endolumenal sealing device in accordance with some embodiments provided herein.

FIG. 4B is a perspective view of another example partially-circumferential endolumenal sealing device in accordance with some embodiments provided herein.

FIG. 4C illustrates another example partially-circumferential endolumenal sealing device implanted in a body vessel to seal a perforation in the wall of the vessel.

FIG. 5A is a perspective view of another example partially-circumferential endolumenal sealing device in accordance with some embodiments provided herein.

FIG. 5B is a perspective view of another example partially-circumferential endolumenal sealing device in accordance with some embodiments provided herein.

FIG. 6 is a perspective view of another example partially-circumferential endolumenal sealing device with auxiliary anchoring features in accordance with some embodiments provided herein.

FIGS. 7A and 7B illustrate a portion of an example partially-circumferential endolumenal sealing device with features for retrieving the device after the device has been deployed in a body.

FIGS. 8A-8F illustrate example configurations of the struts of the partially-circumferential endolumenal sealing devices provided herein.

FIGS. 9A-9I illustrate example configurations of anchoring features that can be used with the partially-circumferential endolumenal sealing devices provided herein.

Like reference symbols in the various drawings indicate like elements. It should also be noted that the accompanying drawing figures referred to herein are not all drawn to scale and may be exaggerated to illustrate various aspects of the present disclosure, and in that regard, the drawing figures should not be construed as limiting.

### DETAILED DESCRIPTION

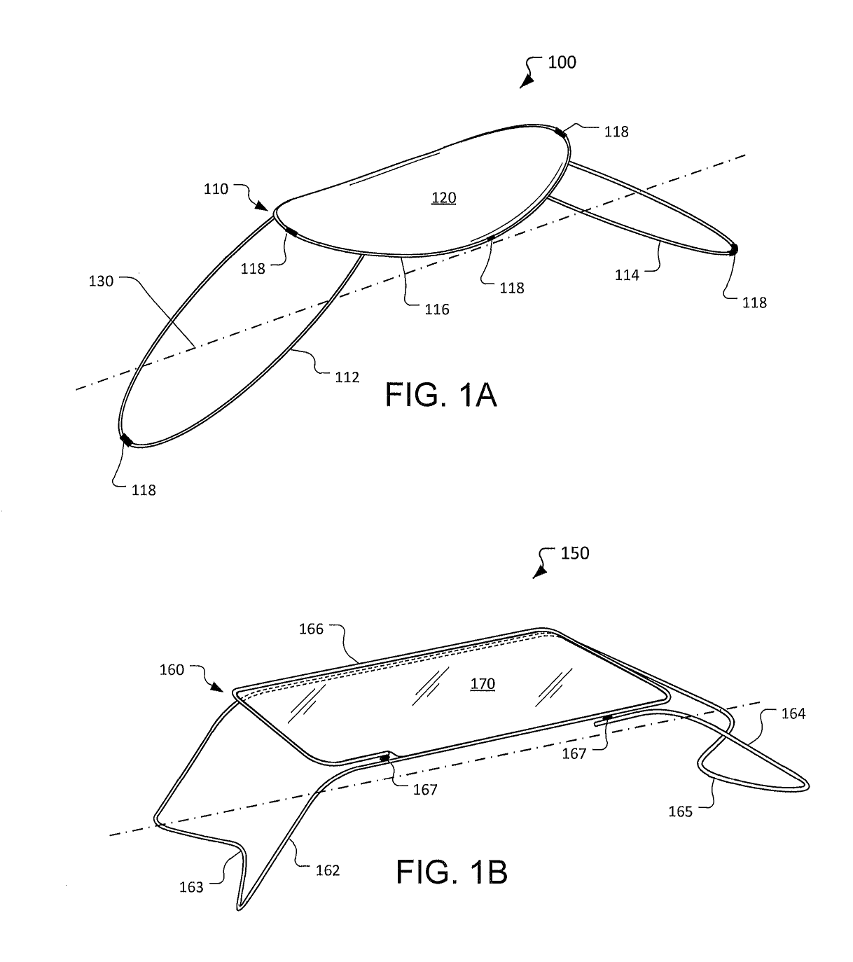

In reference to FIG. 1A, an example embodiment of a partially-circumferential endolumenal sealing device 100 includes framework elements 110 and a covering material 120. In some embodiments, the framework elements 110 include at least a first strut 112, a second strut 114, and a membrane frame 116. The covering material 120 can be disposed on one or more portions of the membrane frame 116, or on all of the membrane frame 116 whereby the covering material 120 substantially covers the entire area defined by the membrane frame 116 (as shown in FIG. 1A).

As will be described in more detail below, the partially-circumferential endolumenal sealing device 100 is configured to be implanted in a patient such that the covering material 120 overlays and seals a defect in a body lumen wall. In that manner, the endolumenal sealing device 100 beneficially restricts the transfer of bio materials through the defect, and thereby promotes healing of the defect by isolating the defect from the contents of the body lumen that otherwise may tend to inhibit the healing process of the tissue surrounding the defect.

The partially-circumferential endolumenal sealing device 100 defines a longitudinal axis 130. In situ, the longitudinal axis 130 of the partially-circumferential endolumenal sealing device 100 approximately coincides with a central axis of the body lumen in which the partially-circumferential endolumenal sealing device 100 is placed.

The framework elements 110 support the covering material 120 in a generally expanded arrangement as shown, and maintain the covering material 120 in a desired location overlaying a defect in a body lumen wall. More specifically, the membrane frame 116 directly supports the covering material 120, and the struts 112 and 114 provide a supporting structure to press the combination of the membrane frame 116 and the covering material 120 against the wall of the body lumen. In this general fashion, the partially-circumferential endolumenal sealing device 100 can be placed in a body lumen to seal a defect in the body lumen wall.

The membrane frame 116 and the covering material 120 make contact with a partial circumferential portion of a body lumen wall. That is, rather than making contact with a full 360° circumference of the body lumen wall, the membrane frame 116 and the covering material 120 make contact with an arcuate portion of the body lumen wall that is less than 360°. For example, in various embodiments the membrane frame 116 and the covering material 120 make contact with an arcuate portion of the body lumen wall of about 120° to 180°, or about 90° to 150°, or about 60° to 120°, or about 30° to 90°, or less than 30°.

In some embodiments, the covering material 120 is made of a membranous material that inhibits or reduces passage of blood, and other bodily fluids and materials. In some embodiments, the covering material 120 has a material composition and configuration that inhibits or prevents tissue ingrowth to the covering material 120. In some embodiments, the covering material 120, or portions thereof, has a microporous structure that provides a tissue ingrowth scaffold for durable sealing and/or supplemental anchoring strength of the sealing device. Some embodiments of the covering material 120 comprise a fluoropolymer, such as an expanded polytetrafluoroethylene (ePTFE) polymer. In some embodiments, the covering material 120 comprises a polyester, a silicone, a urethane, another biocompatible polymer, Dacron, bioabsorbable systems, copolymers, or combinations and subcombinations thereof.

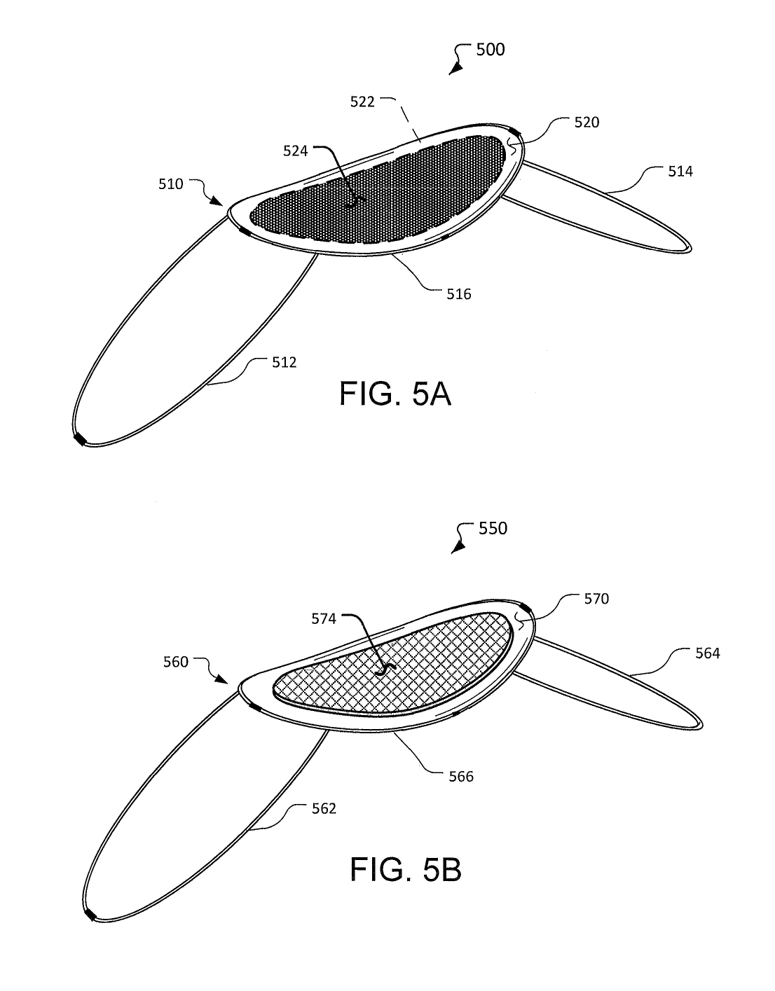

In some embodiments, a first portion of the covering material 120 is formed of a first material and a second portion of the covering material 120 is formed of a second material. For example, as described further in reference to FIGS. 5A and 5B, a central portion of the covering material 120 may be formed of a first material that provides a tissue ingrowth scaffold and is designed to be separated from the sealing device 100 when the sealing device 100 is removed from the body, and the remainder of the covering material 120 may be formed of a second material that inhibits tissue ingrowth and that is designed to remain attached to the sealing device 100 when the sealing device 100 is removed from the body. In some embodiments, portions of the covering material 120 have one or more radiopaque markers attached thereto to enhance in vivo radiographic visualization.

In some embodiments, a periphery of the covering material 120 or at least some edge portions of the covering material 120 are attached to the membrane frame 116. The attachment can be created by a variety of techniques, such as by stitching the covering material 120 to the membrane frame 116, by adhering the covering material 120 to the membrane frame 116, by cohering portions of the covering material 120 around the membrane frame 116, by using clips or barbs, or by other such techniques or combinations thereof. In some embodiments, the membrane frame 116, or portions thereof, may be coated with a bonding agent, for example fluorinated ethylene propylene (FEP) or other suitable adhesive for bonding the covering material 120 to the membrane frame 116. The adhesive may be applied through contact coating, powder coating, dip coating, spray coating, or any other appropriate means. The membrane frame 116 thereby provides a supportive structural framework for the covering material 120 that is otherwise relatively flaccid.

The first and second struts 112 and 114 provide apposition forces to press and maintain the covering material 120 in a desired location overlaying a defect in a body lumen wall. In situ, the first and second struts 112 and 114 make contact with areas of the body lumen wall, and forces are exerted by the first and second struts 112 and 114 onto the body lumen wall at those contact areas. Those forces are also transmitted by the first and second struts 112 and 114 to the membrane frame 116 so as to bias the membrane frame 116 and the covering material 120 into contact with the body lumen wall. In other words, in use the first and second struts 112 and 114 are preloaded and have ongoing mechanical stresses whereby the first and second struts 112 and 114 exert forces to the membrane frame 116 to press the membrane frame 116 and the supported covering material 120 against the body lumen wall to overlay and seal the defect.

The forces applied by the first and second struts 112 and 114 to the membrane frame 116 are not perpendicular to the longitudinal axis 130 of the partially-circumferential endolumenal sealing device 100. Said another way, the first and second struts 112 and 114 apply non-radial apposition forces to the membrane frame 116. Further, the forces exerted by the first and second struts 112 and 114 to the membrane frame 116 can also be described as opposing non-orthogonal forces that press the covering material 120 into contact with the body lumen wall. This design feature of the framework elements 110 eliminates circumferential contact between the body lumen wall and the partially-circumferential endolumenal sealing device 100. In other words, at no transverse cross-section of the body lumen is there a full 360° of circumferential contact with the partially-circumferential endolumenal sealing device 100.

The design of the partially-circumferential endolumenal sealing device 100 facilitates a reliable ongoing seal of a defect in a body lumen wall. That is the case notwithstanding the fact that some anatomical environments in which the endolumenal sealing device 100 are used are dynamic, such as the peristaltic motion of the GI tract. One of the design features that facilitates the reliable ongoing seal of the partially-circumferential endolumenal sealing device 100 is the use of opposing non-orthogonal forces to press the covering material 120 into contact with the body lumen wall.

Some advantages of the designs of the sealing devices provided herein (including the feature of using opposing non-orthogonal forces to press the covering material 120 into contact with the body lumen wall) in the context of the dynamic anatomical environment created by peristalsis will now be explained. Peristaltic motion of the GI tract is comparable to wave motions that propel the contents of the GI tract along the GI tract, such as from the small intestines to the large intestines including the colon. Because, as explained above, at no transverse cross-section of the body lumen is there a full 360° of circumferential contact with the partially-circumferential endolumenal sealing device 100, as a peristaltic wave passes by the sealing device 100, only isolated portions of the framework elements 110 are effected at any one time. In result, therefore, the partially-circumferential endolumenal sealing device 100 will ride the peristaltic wave instead of being pushed along by the wave. In result, the partially-circumferential endolumenal sealing device 100 will maintain a reliable ongoing seal of a defect in a body lumen wall. Further, because no portion of the partially-circumferential endolumenal sealing device 100 is fully-circumferential, the peristaltic motion of the GI tract is not inhibited.

Another design feature of the partially-circumferential endolumenal sealing device 100 that facilitates a reliable ongoing seal of a defect in a body lumen wall is the flexibility and elasticity of the framework elements 110. In general, the framework elements 110 exhibit a high level of flexibility and elasticity. For example, in some embodiments at least some portions of the framework elements 110 are made from super-elastic materials such as nitinol. Further, in some embodiments the framework elements 110 have localized hinge-like areas and other features that are configured to be extra flexible. The flexibility and elasticity of the framework elements 110 allows the partially-circumferential endolumenal sealing device 100 to adapt to the dynamic anatomical environment created by peristalsis without migrating from the desired location within the body lumen. In some embodiments, auxiliary tissue anchorage features are included on the framework elements 110. Such anchorage features can provide increased fixation to resist migration of the partially-circumferential endolumenal sealing device 100 within the body lumen.

The flexibility and elasticity of the framework elements 110 make the partially-circumferential endolumenal sealing device 100 capable of transcatheter deployment. That is, in some embodiments the endolumenal sealing device 100 can be elastically collapsed to a low-profile configuration for temporary containment within a lumen of a delivery catheter or sheath. To deploy the endolumenal sealing device 100, the sheath containing the endolumenal sealing device 100 in the low-profile configuration is inserted into the body of a patient and directed to a target site—typically using radiographic visualization (e.g., fluoroscopy), or using endoscopic optics for direct visualization. At the target site, the endolumenal sealing device 100 is caused to emerge from the sheath, after which the endolumenal sealing device 100 self-expands, or is caused to expand, to an enlarged configuration. For example, FIG. 1A shows the endolumenal sealing device 100 in the enlarged configuration that the endolumenal sealing device 100 will tend to naturally seek in the absence of external constraining forces.

It should be understood that when the endolumenal sealing device 100 is deployed in a patient's body, there will typically be constraining forces applied to the endolumenal sealing device 100, such as from the walls of the body lumen in which the endolumenal sealing device 100 resides. Because of those constraining forces, the shape of the endolumenal sealing device 100 within the body will tend to be different than shown in the figures of the instant specification. Said another way, when the endolumenal sealing device 100 is deployed within the body, the endolumenal sealing device 100 will try to expand to its natural fully enlarged configuration, but the endolumenal sealing device 100 may be constrained by the contours of the anatomy at the target site. In that circumstance, the shape of the endolumenal sealing device 100 will tend to conform to the contours of the anatomy.

After the deployment of the endolumenal sealing device 100 at the target site, the contours of the anatomy may change over time. For example, if the endolumenal sealing device 100 is deployed within the GI tract, the peristaltic wave motion of the intestines may change the contours of the anatomy at the target site. In that circumstance, the flexibility and elasticity of the endolumenal sealing device 100 can allow the framework elements 110 to adapt in shape to thereby facilitate resilient ongoing contact between the covering material 120 and the defect.

As will be described in more detail below, it is envisioned that in some circumstances the endolumenal sealing device 100 will be implanted in a patient for a temporary period of time. This treatment technique may be used, for example, when a defect in a body lumen wall, such as a perforation, needs to be sealed off from the contents of the body lumen and allowed to heal (such as allowing a perforation to heal closed). In some such cases, the endolumenal sealing device 100 and other embodiments described herein can be deployed at the site of the defect and later removed after the perforation has healed. Therefore, in some embodiments the endolumenal sealing device 100 is retrievable, as will be described in more detail later. In addition, in some embodiments portions of the endolumenal sealing device 100 are retrievable while other portions will remain at the defect site. For example, in some embodiments portions of the covering material 120 can provide a scaffold for tissue ingrowth or endothelialization to promote healing of the defect. Then, those portions of the covering material 120 can be made to separate from the endolumenal sealing device 100 and stay at the defect site when the other parts of the endolumenal sealing device 100 are retrieved from the patient's body. In some embodiments, the partially-circumferential endolumenal sealing device 100, or portions thereof, are bioabsorbable such that the structure of the partially-circumferential endolumenal sealing device 100 will deteriorate in time. For example, in some such embodiments portions of the framework elements 110 may deteriorate by bioabsorption, after which other portions of the partially-circumferential endolumenal sealing device 100 may be naturally expelled from the GI tract, or otherwise retrieved.

Still referring to FIG. 1A, in some embodiments the framework elements 110 can be constructed of biocompatible elongate wires having elastic properties that allow for the framework elements 110, and for the overall endolumenal sealing device, to be collapsed to a low-profile configuration for catheter-based delivery or thoracoscopic delivery, and to self-expand to a “memory” induced natural configuration once positioned at a target site within a body. In some embodiments, the endolumenal sealing devices provided herein are also retrievable from the target site within the body using a transcatheter technique, as will be described in more detail below.

In some embodiments, the elastic wire comprising the framework elements 110 may be a spring wire (e.g., L605 steel or stainless steels), shape memory alloy wire (e.g., nitinol or nitinol alloys), super-elastic alloy wire (e.g., nitinol or nitinol alloys), other suitable types of wire, or combinations thereof. In some embodiments, the elastic wire comprising the framework or portions thereof may be constructed of polymeric materials. For example, in some embodiments a polymeric sleeve is used to couple two metallic wires together. In some such embodiments, the polymeric sleeve can provide a living hinge. In some embodiments, different types of wires are used at different locations of the framework elements 110. For example, stainless steel wires can be used for the struts 112 and 114 while nitinol wire is used for the membrane frame 116, or vice versa. In some embodiments, the framework elements 110 are coated with a coating that inhibits thrombus formation.

Features to enhance the radiographic visibility of the partially-circumferential endolumenal sealing devices provided herein are included in some embodiments. For example, in some embodiments the elastic wire framework elements 110 are constructed of a drawn-filled type of nitinol containing a different metal at the core, such as a radiopaque metal (e.g., platinum, tungsten, tantalum, palladium alloys, and the like). Further, in some embodiments, such as with the endolumenal sealing device 100, radiopaque markers 118 are included on portions of the framework elements 110 (or on the covering material 120).

The framework elements 110 of the partially-circumferential endolumenal sealing devices provided herein can comprise one or more wires. For example, the partially-circumferential endolumenal sealing device 100 comprises three discrete wires that are joined together: (1) the first strut 112, (2) the second strut 114, and (3) the membrane frame 116. The wires can be joined together using a variety of techniques including, but not limited to bonding, welding, gluing, by using a sleeve coupling, and the like, and combinations thereof.

In some embodiments, the membrane frame 116 has the two free ends of its elongate wire member joined together to form a closed shape. A variety of shapes are envisioned, such as ovular, circular, rectangular, triangular, and any other suitable shape. The membrane frame 116 can also be three dimensional in shape. For example, the membrane frame 116 can be saddle-shaped or arcuate and have a curvature that approximately corresponds to the curvature of a body lumen wall that the membrane frame 116 will be in contact with. Such a curvature can be induced in the membrane frame 116 using various techniques including, but not limited to, bending, stretching, pressing, stamping, heat-setting, and the like.

In some embodiments, such as the partially-circumferential endolumenal sealing device 100, the two free ends of the first and second struts 112 and 114 are joined to the perimeter of the membrane frame 116 at two locations. As such, the first and second struts 112 and 114 extend from the membrane frame 116. In some embodiments, the first and second struts 112 and 114 each comprise an elongate member that extends from the membrane frame 116 at two or more points and that forms a loop that is shaped as a generally elliptical segment when the sealing device 100 is in a fully expanded configuration. In some embodiments, the first and second struts 112 and 114 are substantially mirror images of each other. In some embodiments, the first and second struts 112 and 114 are dissimilarly sized or shaped in relation to each other.

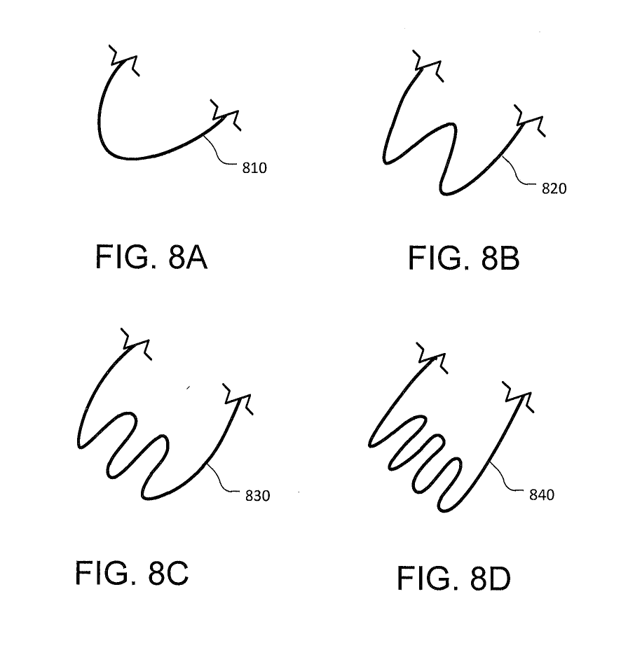

As will be described in more detail below (e.g., in reference to FIGS. 8A-8F), the struts of the partially-circumferential endolumenal sealing devices provided herein can comprise a variety of different features and configurations including, but not limited to linear portions, bends, wavy portions, arcuate portions, sinusoidal portions, eyelets, loops, angular portions, anchorage features, spring-like portions, hinges, and the like, and combinations thereof. While in some embodiments, the framework elements comprise a combination of two or more wires, in some embodiments the framework elements are comprised by a single unitary elongate flexible wire construct.

In reference to FIG. 1B, an example partially-circumferential endolumenal sealing device 150 includes a unitary elongate wire framework element 160 and a covering material 170. The single wire framework element 160 can be formed into a desired shape and joined to itself at appropriate locations 167 to create a first strut 162, a second strut 164, and a membrane frame 166. The joining of the framework element 160 to itself at locations 167 can be performed using the joining techniques described above, e.g., bonding, welding, gluing, using sleeves, and so on. The covering material 170 can be attached to the membrane frame 166 using the techniques described above.

The partially-circumferential endolumenal sealing device 150 can be collapsed to a low-profile configuration for transcatheter delivery to a target site within a body lumen, and for transcatheter retrieval therefrom, as can the other partially-circumferential endolumenal sealing devices provided herein. The partially-circumferential endolumenal sealing device 150 can self-expand, or be assisted to expand, to the configuration shown in FIG. 1B upon emergence from a delivery catheter or sheath. In situ, the configuration of the sealing device 150 will conform to the contours of the anatomy, and may therefore be shaped somewhat differently than shown.

The struts 162 and 164 can have wavy portions 163 and 165 respectively. In some embodiments, such wavy portions 163 and 165 can facilitate the application of an increased amount of non-radial apposition forces to the membrane frame 166. The wavy portions 163 and 165 can also enhance the fixation of the struts 162 and 164 to the body lumen wall.

The membrane frame 166 is generally rectangular. However, a rectangular membrane frame 166 is not a requirement when the framework element 160 is a unitary wire. The membrane frame 166 of the unitary wire framework element 160 can be formed into any shape as desired, such as the other shapes of the membrane frame 116 described above.

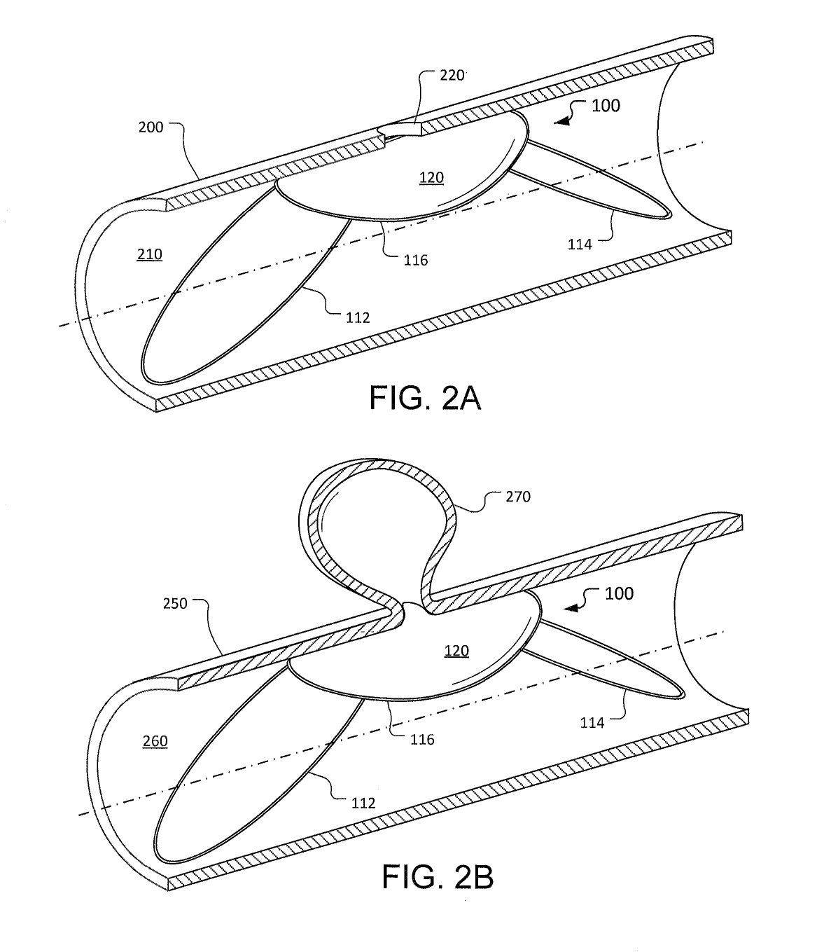

In reference to FIGS. 2A and 2B, the partially-circumferential endolumenal sealing device 100 can be deployed within a body lumen 200 and 250 to treat various types of defects 220 and 270 in the body lumen wall. In general, the struts 112 and 114 of the sealing device 100 contact the inner wall 210 and 260 of the body lumen 200 and 250. The struts 112 and 114 transmit opposing non-orthogonal forces to the membrane frame 116 that press the membrane frame 116 and the covering material 120 against the wall 210 and 260, such that the covering material 120 overlays and seals the defect 220 and 270.

FIGS. 2A and 2B show how the forces applied by the first and second struts 112 and 114 to the membrane frame 116 are not perpendicular to the longitudinal axis 130 of the partially-circumferential endolumenal sealing device 100. That is, the first and second struts 112 and 114 apply non-radial apposition forces to the membrane frame 116 that press the covering material 120 into contact with the body lumen wall 210 and 260. In can also be seen that at no transverse cross-section of the body lumen 200 or 250 is there a full 360° of circumferential contact between the body lumen wall and the partially-circumferential endolumenal sealing device 100.

FIG. 2A depicts a first example use where the partially-circumferential endolumenal sealing device 100 is treating a perforation 220 in the wall of body lumen 200 (shown in cross-section). Such a perforation 220 can be caused by a number of medical situations, such as a resection to remove a lesion, a burst aneurysm, a trauma-induced hole or tear, a fistula, diseases such as appendicitis or diverticulitis, Crohn's disease, and ulcers, to provide a few examples.

The endolumenal sealing devices provided herein can be used to treat perforations of body lumens, such that the perforation is sealed and allowed to heal while being isolated from the contents of the body lumen that may tend to inhibit the healing process. For example, fecal matter within a colon would tend to inhibit the healing process of a perforation of the colon wall. In such circumstances, the partially-circumferential endolumenal sealing device 100 can be temporarily implanted in the colon such that the covering material 120 overlays the perforation of the colon wall. In result, the perforation will be sealed such that fecal matter will not escape from the colon to contaminate other portions of the body, and the tissue surrounding the perforation will be isolated from fecal matter so that the tissue's healing process will not be inhibited. After the perforation has healed closed, the partially-circumferential endolumenal sealing device 100, or portions thereof, can be removed from the colon of the patient.

FIG. 2B depicts a second example use where the partially-circumferential endolumenal sealing device 100 is treating an aneurysm 270 in the wall of body lumen 250 (shown in cross-section). In this example, the body lumen 250 may be a blood vessel such as an artery. An aneurysm is a localized, blood-filled balloon-like bulge in the wall of a blood vessel. Aneurysms are a result of a weakened blood vessel wall, and can be caused by a hereditary condition or an acquired disease. The partially-circumferential endolumenal sealing device 100 can seal off the aneurysm 270 from the lumen of the blood vessel 250 so that the blood pressure within the aneurysm 270 is reduced and the potential for growth or a rupture of the aneurysm 270 is mitigated.

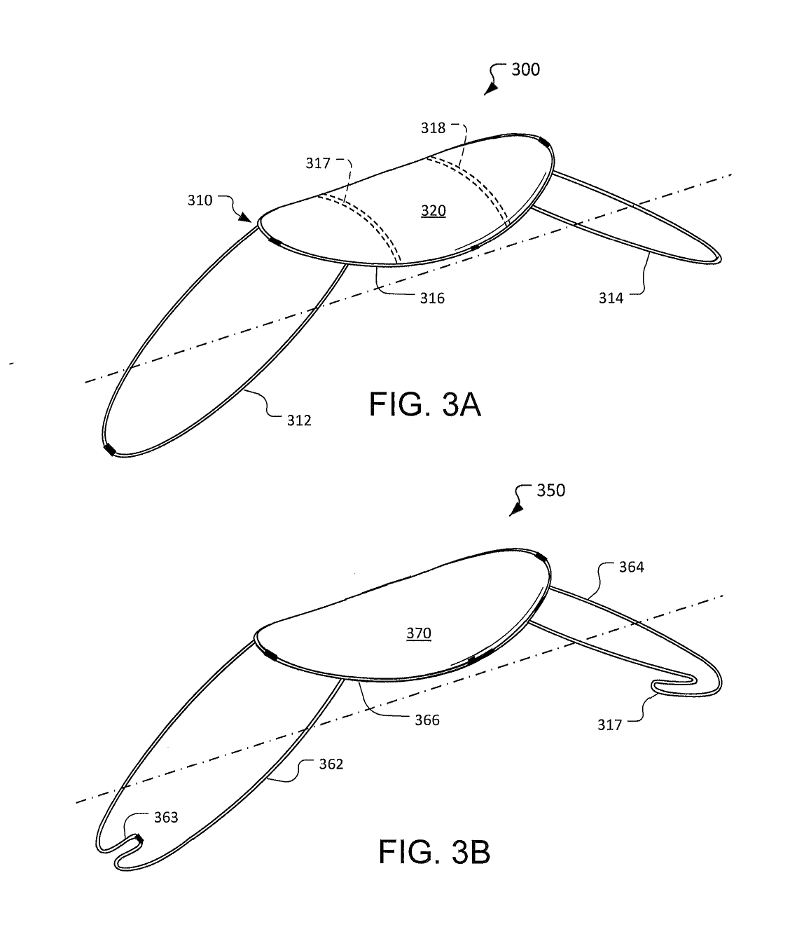

In reference to FIG. 3A, a partially-circumferential endolumenal sealing device 300 includes two elongate cross members 317 and 318 that provide addition support for the covering material 320. Each of the cross members 317 and 318 are joined to the membrane frame 316 between two locations on the perimeter of the membrane frame 316. The cross members 317 and 318 are at least partially located underneath the covering material 320 (in reference to the body lumen) so as to press the covering material 320 into contact with the body lumen wall. The inclusion of cross members 317 and 318 can facilitate resilient ongoing contact between the covering material 320 and the body wall defect being treated.

In some embodiments, the cross members 317 and 318 are arcuately shaped with a curvature that is approximately equal to the curvature of the body lumen wall to facilitate conformance therewith. In some embodiments, the cross members 317 and 318 are arcuately shaped with a greater or lesser degree of curvature in comparison to the curvature of the body lumen wall. In some such embodiments, an additional pressure is applied by the cross members 317 and 318 to the body lumen wall.

The cross members 317 and 318 can be made of the same material as the struts 312 and 314 and the membrane frame 316. Alternatively, the cross members 317 and 318 can be made from a material that is different from the struts 312 and 314 and/or the membrane frame 316. The cross members 317 and 318 can be joined to the membrane frame 316 using the techniques for joining wire members as described elsewhere herein.

In some embodiments, the cross members 317 and 318 are not directly attached to the covering material 320 but rather the cross members 317 and 318 merely support or undergird the covering material 320. In some embodiments, the cross members 317 and 318 are attached to the covering material 320 or portions thereof by a variety of techniques, such as by stitching the covering material 320 to the cross members 317 and 318, by adhering the covering material 320 to the cross members 317 and 318, by using clips or barbs, or by other such techniques, or combinations thereof. In some embodiments, two layers of covering material 320 are adhered to each other and the cross members 317 and 318 are sandwiched between the layers. In some such embodiments, the two layers of covering material 320 can be completely, or at least partially, joined together using an adhesive, stitching, and the like. In some embodiments, the cross members 317 and 318 are woven through the covering material 320 to interlock the cross members 317 and 318 with the covering material 320.

While the partially-circumferential endolumenal sealing device 300 includes two elongate cross members 317 and 318, and the cross members 317 and 318 extend transversely in relation to the longitudinal axis 330, those particular design attributes are not required of all such elongate elements that provide additional support to the covering material 320. For example, in some embodiments one, three, four, or more than four cross members are used. Further, in some embodiments the cross members extend in other directions, such as parallel with the axis 330, or at diagonals thereto. In some embodiments, the cross members extend in two or more directions, such as in a cross-hatched pattern, for example. Some cross members can have a different mechanical properties (such as modulus of elasticity or stiffness) than other cross members, or be comprised of a different material or size of material.

In reference to FIG. 3B, an example partially-circumferential endolumenal sealing device 350 includes struts 362 and 364 that have wavy segment portions 363 and 365 respectively. In some embodiments, the wavy portions 363 and 365 are heat-set into the nonlinear pattern. In some embodiments, the wavy portions 363 and 365 are stretched or formed into the nonlinear pattern. In some embodiments, the wavy portions 363 and 365 can facilitate the application of an increased amount of opposing non-orthogonal apposition forces to the membrane frame 366. The wavy portions 363 and 365 can also enhance the fixation of the struts 362 and 364 to the body lumen wall.

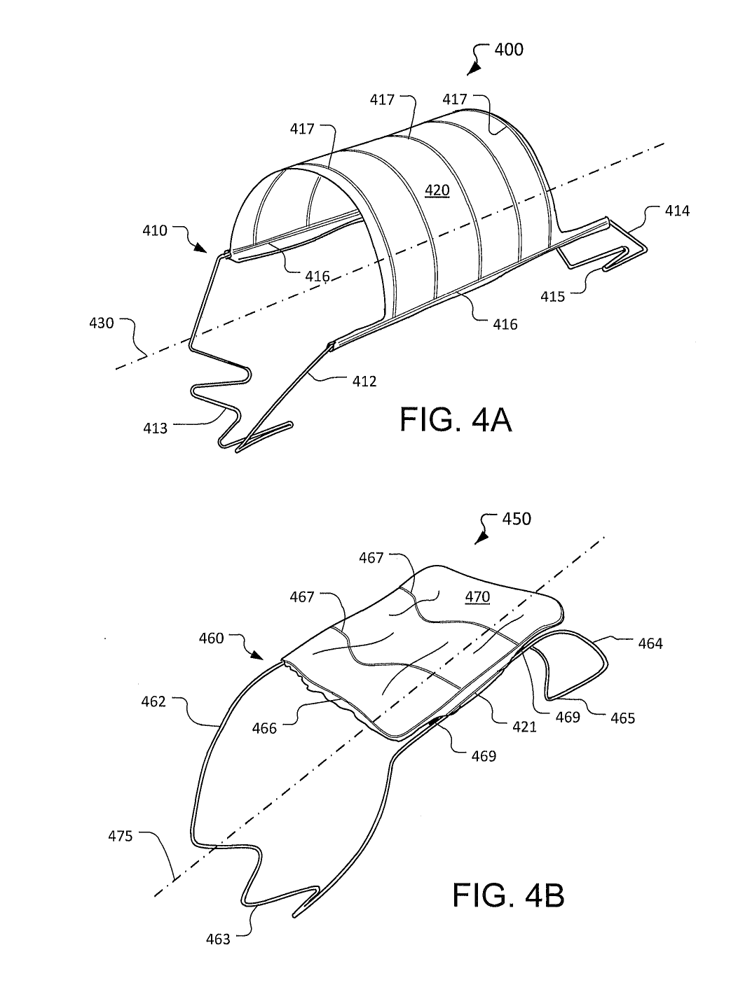

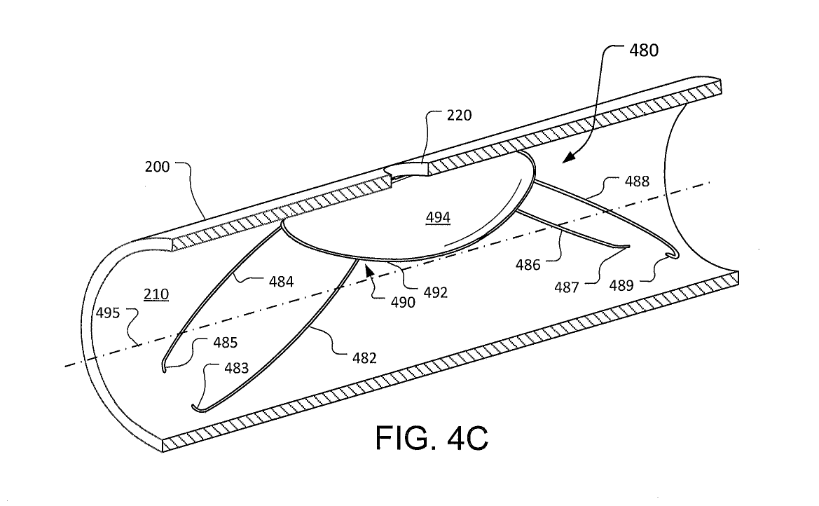

In reference to FIG. 4A, an example embodiment of a partially-circumferential endolumenal sealing device 400 includes framework elements 410 and a covering material 420. In some embodiments, the framework elements 410 include at least a first strut 412, a second strut 414, a membrane frame 416, and one or more cross members 417. The covering material 420 can be disposed on one or more portions of the membrane frame 416 and cross members 417, or on all of the membrane frame 416 and cross members 417 whereby the covering material 420 substantially covers the entire area defined by the membrane frame 416 and cross members 417. The materials and methods of construction of the sealing device 400 are generally analogous to those of the other partially-circumferential endolumenal sealing devices described herein.

The partially-circumferential endolumenal sealing device 400 can be collapsed to a low-profile configuration for transcatheter delivery to a target site within a body lumen, and for transcatheter retrieval therefrom, as can other partially-circumferential endolumenal sealing devices provided herein. The partially-circumferential endolumenal sealing device 400 can self-expand, or be assisted to expand, to the configuration shown in FIG. 4A upon emergence from a delivery catheter or sheath. In situ, the configuration of the sealing device 400 will conform to the contours of the anatomy and may therefore be shaped somewhat differently than shown.

The partially-circumferential endolumenal sealing device 400 includes a membrane frame 416 with two elongate wire members that are generally parallel with the axis 430 and to which the covering material 420 is attached. In some embodiments, the covering material 420 can be reinforced by one or more elongate cross members, such as the five cross members 417 of the endolumenal sealing device 400. The two elongate wire members of the membrane frame 416 can be combined with the cross members 417 to define a rectangular arcuate framework on which the covering material 420 is disposed.

In this example, the partially-circumferential endolumenal sealing device 400 is configured to contact an arcuate portion of a body lumen wall of about 180°. A similar type of construction can be used to create other partially-circumferential endolumenal sealing devices that have different proportions. For example, some embodiments of partially-circumferential endolumenal sealing devices include an arcuate rectangular framework that is configured to contact an arcuate portion of a body lumen wall of about 120° to 180°, or about 90° to 150°, or about 60° to 120°, or about 30° to 90°, or less than 30°.

In some embodiments, the struts 412 and 414 include wavy segment portions 413 and 415 respectively. In some embodiments, other types of wavy portions or no such wavy portions are included in the struts 412 and 414. The struts 412 and 414 are configured to exert opposing non-orthogonal apposition forces to the membrane frame 416, to thereby press the covering material 420 against the wall of a body lumen, and to overlay and seal a defect in the body lumen wall.

In reference to FIG. 4B, an example embodiment of a partially-circumferential endolumenal sealing device 450 includes framework elements 460 and a covering material 470. In some embodiments, the framework elements 460 include at least a first strut 462, a second strut 464, a membrane frame 466, and one or more cross members 467. The covering material 470 can be disposed on one or more portions of the membrane frame 466 and cross members 467, or on all of the membrane frame 466 and cross members 467 whereby the covering material 470 substantially covers the entire area defined by the membrane frame 466 and cross members 467. The materials and methods of construction of the sealing device 450 are generally analogous to those of the other partially-circumferential endolumenal sealing devices described herein.

The partially-circumferential endolumenal sealing device 450 can be collapsed to a low-profile configuration for transcatheter delivery to a target site within a body lumen and for transcatheter retrieval therefrom, as can other partially-circumferential endolumenal sealing devices provided herein. The partially-circumferential endolumenal sealing device 450 can self-expand, or be assisted to expand, to the configuration shown in FIG. 4B upon emergence from a delivery catheter or sheath. In situ, the configuration of the sealing device 450 will conform to the contours of the anatomy and may therefore be shaped somewhat differently than shown.

The membrane frame 466 is substantially rectangular, as defined by two elongate wire members that are generally parallel with the axis 475 and four cross members 467. In some embodiments, the membrane frame 466 can incorporate other shapes and constructions of elongate wire frame members, combinations of different types of frame members, hinge points, and the like. The membrane frame 466 is substantially planar. In some embodiments, the membrane frame 466 can be arcuate as described elsewhere herein (e.g., refer to FIG. 4A). One or more of the cross members 467 can be non-linear, such as wavy, sinusoidal, saw-tooth patterned, and the like. In some embodiments, some of the one or more cross members 467 are shaped differently than the others of the one or more cross members 467.

In some embodiments, the partially-circumferential endolumenal sealing device 450 can include two elongate elements 421 that are generally parallel with the axis 475 and that interconnect the struts 462 and 464. In some embodiments, a unitary wire comprises the two elongate elements 421 and the struts 462 and 464. In some embodiments, two or more wires are joined together to comprise the two elongate elements 421 and the struts 462 and 464.

The membrane frame 466 can be joined to the two elongate elements 421 that interconnect the struts 462 and 464. For example, in some embodiments, the membrane frame 466 is joined to each of the two elongate elements 421 at two joints 469 as shown. In some embodiments, instead of two joints 469, the membrane frame 466 is joined to each of the two elongate elements 421 by a single joint, an elongated joint, or by three or more joints. In some embodiments, the two joints 469 can function as hinge points. In some such cases, the end portions of the two elongate elements 421 can be flexible and thereby pivotable in relation to the middle portion of the elongate elements 421 that is joined to the membrane frame 466. Such an ability by the elongate elements 421 to pivot can increase the overall flexibility of the partially-circumferential endolumenal sealing device 450, thereby enhancing the device's ability to conform with the contours of the anatomy. In some embodiments, hinge portions are included on the membrane frame 466 to also enhance the device's ability to conform with the contours of the anatomy.

In reference to FIG. 4C, an example embodiment of a partially-circumferential endolumenal sealing device 480 includes framework elements 490 and a covering material 494. The sealing device 480 can be used, for example, to seal a perforation 220 in a wall 210 of a body lumen 200. In some embodiments, the framework elements 490 include at least a first strut 482, a second strut 484, a third strut 486, a fourth strut 488, and a membrane frame 492. The covering material 494 can be disposed on one or more portions of the membrane frame 492, or on all of the membrane frame 492 whereby the covering material 494 substantially covers the entire area defined by the membrane frame 492. The materials and methods of construction of the sealing device 480 are generally analogous to those of the other partially-circumferential endolumenal sealing devices described herein. While the illustrated embodiment includes four struts 482, 484, 486, and 488, in some embodiments other numbers of struts are included. For example, in some embodiments one, two, three, five, six, or more than six struts are included.

The struts 482, 484, 486, and 488 of the partially-circumferential endolumenal sealing device 480 are elongate wire members that each include a first and a second end. The first ends of the struts 482, 484, 486, and 488 terminate at the membrane frame 492 such that the struts 482, 484, 486, and 488 each extend from the membrane frame 492. The second ends of the struts 482, 484, 486, and 488 are free ends 483, 485, 487, and 489 respectively. Therefore, each strut 482, 484, 486, and 488 is an individual supporting member or leg that extends from the membrane frame 492.

The forces applied by the struts 482, 484, 486, and 488 to the membrane frame 492 are not perpendicular to the longitudinal axis 495 of the partially-circumferential endolumenal sealing device 480. Said another way, the struts 482, 484, 486, and 488 apply non-radial apposition forces to the membrane frame 492. Further, the forces exerted by the struts 482, 484, 486, and 488 to the membrane frame 492 can also be described as opposing non-orthogonal forces that press the covering material 494 into contact with the body lumen wall 210. This design feature of the framework elements 490 eliminates circumferential contact between the body lumen wall 210 and the partially-circumferential endolumenal sealing device 480. In other words, at no transverse cross-section of the body lumen 200 is there a full 360° of circumferential contact with the partially-circumferential endolumenal sealing device 480.

As mentioned previously, the struts 482, 484, 486, and 488 of the partially-circumferential endolumenal sealing device 480 include free ends 483, 485, 487, and 489 respectively. In some embodiments, the free ends 483, 485, 487, and 489 are configured as atraumatic ends. For example, the free ends 483, 485, 487, and 489 can be formed to have blunt ends or feet portions that inhibit penetration of the free ends 483, 485, 487, and 489 into the body lumen wall 210. In some embodiments, the free ends 483, 485, 487, and 489 can be configured to penetrate into the body lumen 200 to enhance the anchorage of the sealing device 480 in relation to the body lumen 200.

As with other struts described herein, some or all of the struts 482, 484, 486, and 488 can comprise a variety of different features and configurations including, but not limited to, linear portions, bends, wavy portions, arcuate portions, sinusoidal portions, eyelets, loops, angular portions, anchorage features, spring-like portions, hinges, and the like, and combinations thereof. In some embodiments, the struts 482, 484, 486, and 488 each have the same general shape and features. In some embodiments, one or more of the struts 482, 484, 486, and 488 is different than the others. For example, a particular one or more of the struts 482, 484, 486, or 488 can have a shape and/or features that the others of the struts 482, 484, 486, or 488 do not have.

In reference to FIGS. 5A and 5B, the example partially-circumferential endolumenal sealing devices 500 and 550 (and others embodiments provided herein) can include portions 524 and 574 of the covering material 520 and 570 that are configured to remain in the body lumen of a patient after the other portions of the endolumenal sealing devices 500 and 550 have been retrieved/removed. In some embodiments, the portions 524 and/or 574 are made from a different material than the other portions of the covering material 520 and 570, while in some embodiments the materials of the portions 524 and/or 574 and the covering material 520 and 570 are the same.

In a first example, in reference to FIG. 5A, the covering material 520 used for the partially-circumferential endolumenal sealing device 500 includes a detachable portion 524. In some embodiments, the detachable portion 524 can separate from the other portion(s) of the covering material 520 by virtue of a discontinuity 522 that provides a weakened separation interface between the detachable portion 524 and the other portion(s) of the covering material 520. In some embodiments, the discontinuity 522 is a perforation. In some embodiments, the portion 524 does not detach from the other portion(s) of the covering material 520. In some detachable embodiments, other techniques for weakening the covering material 520 at the discontinuity 522 are used, e.g., heat embossing, abrading, and the like. In some embodiments, bioabsorbable clips or sutures are used to join together the detachable portion 524 and the other portion(s) of the covering material 520 at the discontinuity 522. In situ, the bioabsorbable clips or sutures can dissolve to thereby detach the detachable portion 524 from the other portion(s) of the covering material 520, so that the detachable portion 524 will remain in the body lumen after the other portions of the partially-circumferential endolumenal sealing device 500 have been removed. It should be recognized that the shape of the detachable portion 524, while illustrated as ovular, can be a variety of different shapes including circular, rectangular, square, triangular, and the like.

In a second example, in reference to FIG. 5B, the covering material 570 used for the partially-circumferential endolumenal sealing device 550 includes an additional pad 574. In some embodiments, the pad 574 is positioned on the surface of the covering material 570 such that there are two layers of materials where the pad 574 is located. In some embodiments, the pad 574 can separate from the covering material 570 by virtue of bioabsorbable sutures, bioabsorbable adhesives, removable stitching, and the like. Accordingly, the pad 574 can remain in the body lumen after the other portions of the partially-circumferential endolumenal sealing device 500 have been removed. In some embodiments, the pad 574 remains attached to the coving material 570. It should be recognized that the shape of the pad 574, while illustrated as ovular, can be a variety of different shapes including circular, rectangular, square, triangular, and the like.

In some embodiments, the covering materials, detachable portions, or pad materials used in the endolumenal sealing devices provided herein are modified by one or more chemical or physical processes that enhance certain properties of the materials. For example, in some embodiments, a hydrophilic coating is applied to the materials to improve the wettability and echo translucency of the materials. In some embodiments the covering materials, detachable portions, or pad materials are modified with chemical moieties that promote one or more of endothelial cell attachment, endothelial cell migration, endothelial cell proliferation, and resistance to or promotion of thrombosis. In some embodiments the covering materials, detachable portions, or pad materials are modified with one or more covalently attached drug substances (e.g., heparin, antibiotics, and the like) or impregnated with the one or more drug substances. The drug substances can be released in situ to promote healing, reduce tissue inflammation, reduce or inhibit infections, and to promote various other therapeutic treatments and outcomes. In some embodiments the drug substance is a corticosteroid, a human growth factor, an anti-mitotic agent, an antithrombotic agent, a stem cell material, or dexamethasone sodium phosphate, to name some examples.

Coatings and treatments may be applied before or after the covering materials, detachable portions, or pad materials are joined or disposed on the wire members of the endolumenal sealing devices. Additionally, one or both sides of the covering materials, detachable portions, or pad materials may be coated. In some embodiments, certain coatings and/or treatments are applied to the material(s) located on some portions of an endolumenal sealing device, and other coatings and/or treatments are applied to the material(s) located on other portions of the endolumenal sealing devices. In some embodiments, a combination of multiple coatings and/or treatments are applied to the covering materials, detachable portions, or pad materials. In some embodiments, certain portions of the device are left uncoated and/or untreated.

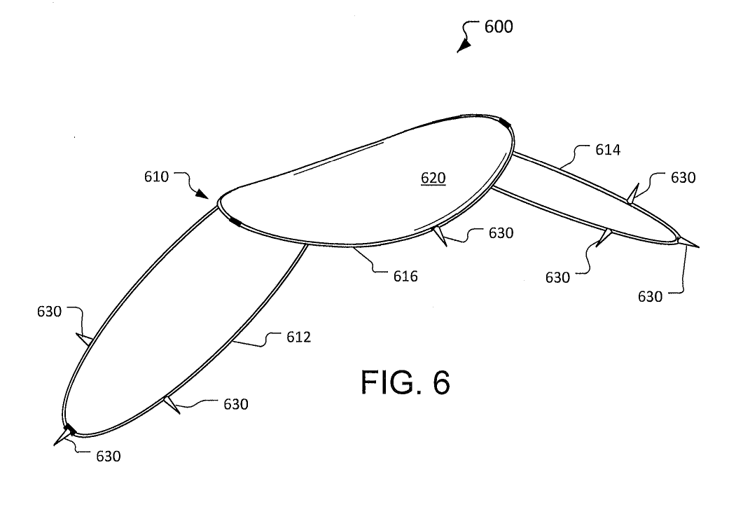

In reference to FIG. 6, an example partially-circumferential endolumenal sealing device 600 includes framework elements 610 and a covering material 620. In some embodiments, the framework elements 610 include at least a first strut 612, a second strut 614, a membrane frame 616, and one or more example anchor features 630. Such anchor features of various types and configurations may be attached to any of the partially-circumferential endolumenal sealing device embodiments provided herein, if so desired.

The partially-circumferential endolumenal sealing device 600 can be collapsed to a low-profile configuration for transcatheter delivery to a target site within a body lumen and for transcatheter retrieval therefrom, as can other partially-circumferential endolumenal sealing devices provided herein. The partially-circumferential endolumenal sealing device 600 can self-expand, or be assisted to expand, to the configuration shown in FIG. 6 upon emergence from a delivery catheter or sheath. In situ, the configuration of the sealing device 600 will conform to the contours of the anatomy and may therefore be shaped somewhat differently than shown.

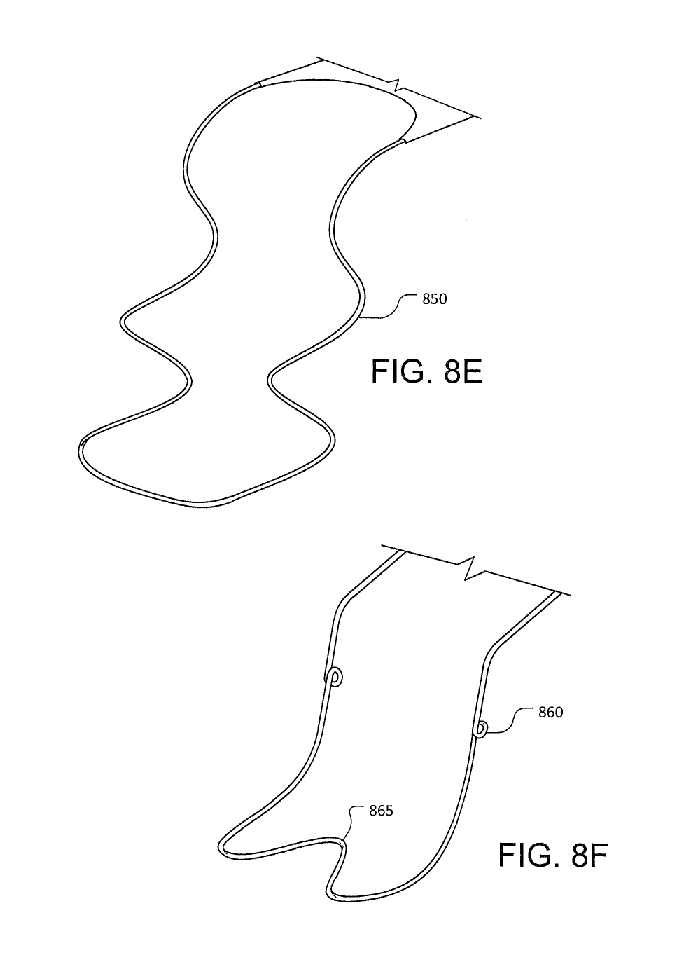

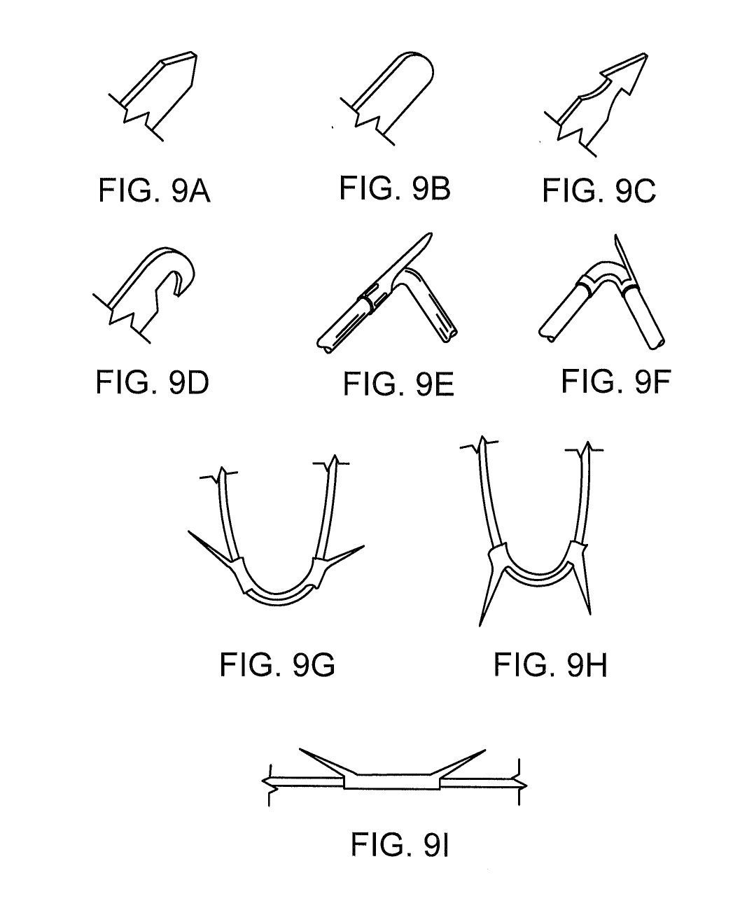

As exemplified by the partially-circumferential endolumenal sealing device 600, in some embodiments the partially-circumferential endolumenal sealing devices provided herein include various types of fixation anchors. Some non-limiting examples of various types of fixation anchors are provided in FIGS. 9A through 9I. Fixation anchors can contact, and in some cases pierce, surrounding tissue at a target deployment site so as to secure the position of the device, or certain portions of the device, at the target deployment site. For example, the example partially-circumferential endolumenal sealing device 600 includes fixation anchor features 630 on the membrane frame 616 and on the struts 612 and 614. In some embodiments fewer, more, or no fixation anchor features are provided. Fixation anchors can be made from a variety of suitable materials. For example, the fixation anchor features can be made of nitinol, L605 steel, stainless steel, a polymeric material, or any other appropriate biocompatible material. In some embodiments, the fixation anchor features can be made from a non-permanent biodegradable or bioabsorbable material. The super-elastic properties of nitinol make it a good material for such fixation anchor features. Nitinol can be heat-set so that a fixation anchor can self-expand into a desired shape when the fixation anchor is placed in a less restrictive environment, such as when it is deployed from the delivery sheath to a body lumen. In some embodiments, it is desirable for a fixation anchor feature to be biased to have a particular shape to enhance the anchoring properties of the fixation anchor.

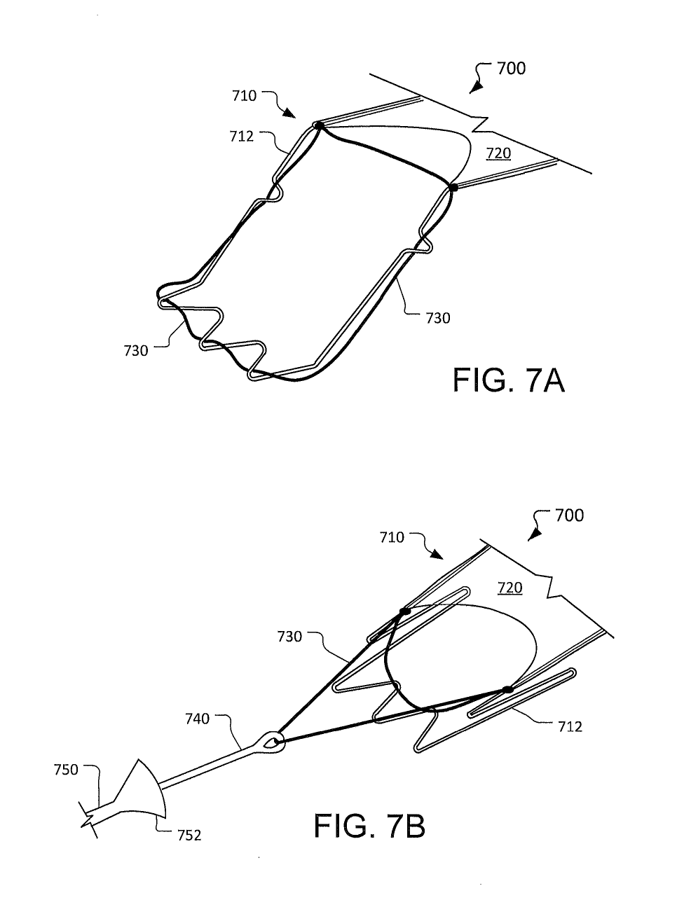

In reference to FIGS. 7A and 7B, an example partially-circumferential endolumenal sealing device 700 that has been previously deployed in a body lumen can be retrieved from the body lumen by tensioning a retrieval cord 730 that is engaged with a strut 712 of the endolumenal sealing device 700. Applying tension to the retrieval cord 730, using a grasping tool 740, can cause the endolumenal sealing device 700 to collapse to a lower-profile configuration for insertion in a retrieval sheath 750. Retrieval features of various types and configurations, such as the retrieval cord 730, may be included with any of the partially-circumferential endolumenal sealing device embodiments provided herein, if so desired.

The retrieval cord 730 can be slidably coupled with the strut 712 at various locations on the strut 712. In some such locations on the strut 712, eyelets, bends, loops, and the like, can be used to facilitate the coupling between the strut 712 and the retrieval cord 730. As shown in FIG. 7A, the retrieval cord 730 can be remain coupled to the strut 712 when the endolumenal sealing device 700 is in use in a body lumen. In some embodiments, the retrieval cord 730 can be made of a polymer material including, but not limited to, nylon, polypropylene, PTFE, silk, and the like. In some embodiments, the retrieval cord can be made of a metallic material including, but not limited to, nitinol, aluminum, stainless steel, and the like. The retrieval cord 730 can be a monofilament or braided and the like.

When retrieval of the endolumenal sealing device 700 from the body lumen is desired, a retrieval sheath 750 containing a grasping tool 740 can be routed to the location of the endolumenal sealing device 700 in the patient's body. The grasping tool 740 can be used to couple with the retrieval cord 730. As the grasping tool 740 is thereafter retracted, tension is applied to the retrieval cord 730. The tensioning and displacement of the retrieval cord 730 caused by the grasping tool 740 will cause the strut 712 to collapse as illustrated in FIG. 7B, and the other portions of the endolumenal sealing device 700 may also collapse to some extent. As the grasping tool 740 is retracted further, including to within the retrieval sheath 750, the strut 712 will be drawn into the distal end of the retrieval sheath 750. A funnel 752 can be included on the distal end of the retrieval sheath 750. The funnel 752 will provide an wider initial opening at the distal tip of the retrieval sheath 750 to facilitate the capture of all portions of the strut 712. As the grasping tool 740 is further retracted, the entire endolumenal sealing device 700 can be pulled into the lumen of the retrieval sheath 750. Then the retrieval sheath 750, containing the endolumenal sealing device 700, can be removed from the patient.

In reference to FIGS. 8A through 8F, various features that can be incorporated into the configuration of the struts for use as desired in some embodiments of the partially-circumferential endolumenal sealing devices provided herein are illustrated. It should be understood that these figures do not provide an exhaustive compilation of features that can be incorporated into the configuration of the struts. But rather, these figures provide examples of the types of configurations that are envisioned.

FIG. 8A depicts an apex portion of a strut comprised of an elliptical segment 810. FIG. 8B depicts an apex portion of a strut having a single waveform 820. FIG. 8C depicts an apex portion of a strut having a double waveform 830. FIG. 8D depicts an apex portion of a strut having a triple waveform 840. FIG. 8E depicts a portion of a strut that has a wavy patterns 850 in the elongate side portions of the strut. FIG. 8F depicts a portion of a strut that has loops 860 in the elongate side portions of the strut and a single waveform 865 in the apex of the strut.

In comparison to the elliptical segment of FIG. 8A, the configurations of FIGS. 8B-8D will generally tend to increase the fixation and resistance to migration of the endolumenal sealing devices. In comparison to a strut with linear or generally linear elongate side portions, the configurations of FIGS. 8E and 8F will add flexibility (e.g., spring-like regions and hinge points) to the strut to enhance the ability of the endolumenal sealing devices to conform with the contours of the anatomy.

In reference to FIGS. 9A through 9I, a variety of example anchor feature tissue engagement tips that can be incorporated into the configurations of the partially-circumferential endolumenal sealing devices provided herein as desired are illustrated. As described previously, in some implementations, an anchor feature comprising a tissue engagement member configured to penetrate tissue may be desired. In other implementations, an anchor feature comprising a tissue engagement member with a blunt tip that is configured to make contact with the tissue but not penetrate the tissue may be desired. Still further types of special tip configurations are also envisioned. A single endolumenal sealing device may strategically use a combination of various types of anchor feature configurations at various locations on the framework of the device. Anchor features, or fixation devices may be attached to any of the partially-circumferential endolumenal sealing device embodiments provided herein, if so desired.

In some embodiments, the anchor features are joined directly to the wire members of the struts or membrane frame, such as by welding or gluing. In some embodiments, the anchor features comprise a tube that is cut (e.g., laser cut) and the tube is placed over the wire member of the struts or membrane frame.

FIG. 9A illustrates a type of a tissue engagement tip that may be intended to penetrate surrounding tissue. The edges of the tip may also be sharpened to facilitate penetration. Whereas, in the illustrated embodiment, the apex of the tip is approximately on the centerline of the tip, in some embodiments, the apex of the tip may be in alignment with an edge of the tissue engagement member. In some fixation anchor implementations, it can be desired to be able to relatively atraumatically remove the tissue engagement members from a penetrated tissue to reposition or remove the partially-circumferential endolumenal sealing devices provided herein. The embodiment shown may facilitate relatively convenient removal of the tip from tissue after it has penetrated the tissue, for example, because the tip does not include a barb.

FIG. 9B illustrates a type of a tissue engagement tip provides a blunt tip that may contact tissue but may avoid penetrating the tissue. In some implementations, it may be desirable to provide some anchoring strength without piercing the surrounding tissue. A blunt tip as shown may enable such an implementation. Tips with additional surface area at a tissue contact region (e.g., a “paddle” shape or a “shoe” shape) can also be used.

FIGS. 9C and 9D illustrate types of tissue engagement tips that may be intended to penetrate tissue, and to provide a substantial anchoring strength—even in the direction linearly opposite to the penetration direction. These tip embodiments therefore include one or more barbed portions. As a result, when a tissue engagement member with this type of tip has penetrated tissue, the barbed portions may tend to resist removal because the edges of the barbed portions will make contact with internal tissue so that the tissue will interfere with the tip's removal. Many other styles of barbed tip embodiments are also envisioned.

FIGS. 9E and 9E illustrate types of anchor features that are made of tubes that have been cut to create a single tissue engagement prong and a collar for mounting the tube on a wire. These anchor features can be used at bent areas of the wire members. FIGS. 9G and 9H illustrate types of tissue engagement anchor features that are made of tubes that have been cut to create twin tissue engagement prongs and a collar for mounting the tube on a wire. These anchor features can also be used at bent areas of the wire members. FIG. 9I illustrates a type of anchor feature that is made of a tube that has been cut to create twin tissue engagement prongs and a collar for mounting the tube on a wire. This anchor feature can be used at a generally linear area on a wire member.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In addition to being directed to the teachings described above and claimed below, devices and/or methods having different combinations of the features described above and claimed below are contemplated. As such, the description is also directed to other devices and/or methods having any other possible combination of the dependent features claimed below.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

What is claimed is:

1. An implantable medical device for treating a portion of a body lumen wall, the device comprising:
a membrane frame;
a membrane material disposed on at least a portion of the membrane frame;
a first strut extending from the membrane frame; and
a second strut extending from the membrane frame, wherein the first and second struts are configured to exert opposing non-orthogonal forces to the membrane frame to thereby press at least a portion of the membrane material into contact with the body lumen wall.

2. The device of claim 1, wherein the portion of the body lumen wall comprises a defect and the membrane material is configured to overlay the defect.

3. The device of claim 1, wherein the first and second struts each comprise elongate members that are unitary with an elongate member of the membrane frame.

4. The device of claim 1, wherein the first and second struts each comprise elongate members that are joined to an elongate member of the membrane frame.

5. The device of claim 1, wherein the first and second struts each comprise elongate members that are joined to an elongate member of the membrane frame by one or more of a weld, an adhesive, and a sleeve.

6. The device of claim 1, wherein the first and second struts extend from a perimeter of the membrane frame.

7. The device of claim 1, wherein the first and second struts each comprise a discrete elongate member that is attached to the membrane frame at two or more points, and that forms a loop that is shaped as a generally elliptical segment when the device is in a fully expanded configuration.

8. The device of claim 1, wherein the first and second struts have substantially identical shapes when the device is in a fully expanded configuration.

9. The device of claim 1, wherein the membrane material covers substantially the entire area defined by the membrane frame.

10. The device of claim 1, wherein the device assumes a low-profile configuration when the device is maintained in a constraining environment, and wherein the device expands from the low-profile configuration and assumes an expanded configuration when the device is liberated from the constraining environment.

11. The device of claim 1, wherein the membrane frame is configured to contact an arcuate portion of the body lumen wall of about 120 to 180 degrees.

12. The device of claim 1, wherein the membrane frame is configured to contact an arcuate portion of the body lumen wall of about 60 to 120 degrees.

13. The device of claim 1, wherein one or more of the first and second struts include hinge points such that the one or more of the first and second struts are pivotable in relation to the membrane frame.

14. The device of claim 1, wherein the membrane material is configured to inhibit tissue ingrowth and endothelialization into the membrane material.

15. The device of claim 1 wherein at least a portion of the membrane material is configured to be separated from the device and remain in a body when the device is removed from the body after treating the portion of the body lumen wall.

16. The device of claim 15, further comprising a bioabsorbable component that facilitates the separation of the at least a portion of the membrane material from the device.

17. The device of claim 15, wherein at least a portion of the membrane material that is configured to be separated from the device is configured to promote tissue ingrowth or endothelialization into the membrane material.

18. The device of claim 17, wherein the portion of the membrane material that is configured to promote tissue ingrowth or endothelialization into the membrane material has an average porosity in the range of about 20 to 250 microns.

19. The device of claim 17, wherein the portion of the membrane material that is configured to promote tissue ingrowth or endothelialization into the membrane material has an average porosity in the range of about 100 to 200 microns.

20. The device of claim 17, wherein the portion of the membrane material that is configured to promote tissue ingrowth or endothelialization into the membrane material includes a plurality of openings that have an average diameter of about 0.25 to 2.0 millimeters.

21. The device of claim 17, wherein the portion of the membrane material that is configured to promote tissue ingrowth or endothelialization into the membrane material includes a coating of one or more growth factors.

22. The device of claim 1, further comprising an attachment feature configured for releasably coupling with a delivery device or a retrieval device.

23. The device of claim 1, wherein the membrane frame further comprises one or more elongate elements extending between two locations on a perimeter of the membrane frame.

24. The device of claim 23, wherein the one or more elongate elements extending between two locations on the perimeter of the membrane frame are non-linear.

25. The device of claim 1, further comprising one or more radiopaque markers on the membrane frame.

26. The device of claim 1, further comprising one or more radiopaque markers on one or more of the first and second struts.

27. The device of claim 1, wherein the portion of the body lumen wall comprises a defect and wherein the device is configured to prevent protrusion of the membrane material into the defect.

28. The device of claim 1, wherein the membrane frame has a length of about 0.5 to 15 centimeters.

29. The device of claim 1, further comprising one or more tissue anchorage features on the membrane frame, on one or more of the first and second struts, or on both the membrane frame and one or more of the first and second struts.

30. The device of claim 1, wherein the struts include atraumatic end portions.

31. The device of claim 1, wherein at least portions of the first and second struts or the membrane frame are coated with a coating that inhibits thrombus formation.

32. The device of claim 1, wherein at least a portion of the membrane material is modified by one or more chemical or physical processes to enhance particular properties of the materials.

33. An implantable medical device for treating a portion of a body lumen wall, the device comprising:
a membrane frame;
a membrane material disposed on at least a portion of the membrane frame;
a first strut extending from the membrane frame; and
a second strut extending from the membrane frame, wherein the first and second struts are configured to press at least a portion of the membrane material into contact with the body lumen wall, and wherein the first and second struts each comprise an elongate member that extends from the membrane frame at two or more points and that forms a loop that is shaped as a generally elliptical segment when the device is in a fully expanded configuration.

34. The device of claim 33, wherein the portion of the body lumen wall comprises a defect and the membrane material is configured to overlay the defect.

35. The device of claim 33, wherein the first and second struts each comprise elongate members that are unitary with an elongate member of the membrane frame.

36. The device of claim 33, wherein the first and second struts each comprise elongate members that are joined to an elongate member of the membrane frame.

37. The device of claim 33, wherein the first and second struts each comprise elongate members that are joined to an elongate member of the membrane frame by one or more of a weld, an adhesive, and a sleeve.

38. The device of claim 33, wherein the first and second struts extend from a perimeter of the membrane frame.

39. The device of claim 33, wherein the first and second struts have substantially identical shapes when the device is in a fully expanded configuration.

40. The device of claim 33, wherein the membrane material covers substantially the entire area defined by the membrane frame.

41. The device of claim 33, wherein the device assumes a low-profile configuration when the device is maintained in a constraining environment, and wherein the device expands from the low-profile configuration and assumes an expanded configuration when the device is liberated from the constraining environment.

42. The device of claim 33, wherein the membrane frame is configured to contact an arcuate portion of the body lumen wall of about 120 to 180 degrees.

43. The device of claim 33, wherein the membrane frame is configured to contact an arcuate portion of the body lumen wall of about 60 to 120 degrees.

44. The device of claim 33, wherein the membrane material is configured to inhibit tissue ingrowth and endothelialization into the membrane material.

45. The device of claim 33, wherein at least a portion of the membrane material is configured to be separated from the device and remain in a body when the device is removed from the body after treating the portion of the body lumen wall.

46. The device of claim 45, wherein at least a portion of the membrane material that is configured to be separated from the device is configured to promote tissue ingrowth or endothelialization into the membrane material.

47. The device of claim 46, wherein the portion of the membrane material that is configured to promote tissue ingrowth or endothelialization into the membrane material has an average porosity in the range of about 20 to 250 microns.

48. The device of claim 46, wherein the portion of the membrane material that is configured to promote tissue ingrowth or endothelialization into the membrane material has an average porosity in the range of about 100 to 200 microns.

49. The device of claim 46, wherein the portion of the membrane material that is configured to promote tissue ingrowth or endothelialization into the membrane material includes a plurality of openings that have an average diameter of about 0.25 to 2.0 millimeters.

50. The device of claim 46, wherein the portion of the membrane material that is configured to promote tissue ingrowth or endothelialization into the membrane material includes a coating of one or more growth factors.

51. The device of claim 33, further comprising an attachment feature configured for releasably coupling with a delivery device or a retrieval device.

52. The device of claim 33, wherein the membrane frame further comprises one or more elongate elements extending between two locations on a perimeter of the membrane frame.

53. The device of claim 52, wherein the one or more elongate elements extending between two locations on the perimeter of the membrane frame are non-linear.

54. The device of claim 33, further comprising one or more radiopaque markers on the membrane frame.

55. The device of claim 33, further comprising one or more radiopaque markers on one or more of the first and second struts.

56. The device of claim 33, wherein the portion of the body lumen wall comprises a defect and wherein the device is configured to prevent protrusion of the membrane material into the defect.

57. The device of claim 33, wherein the membrane frame has a length of about 0.5 to 15 centimeters.

58. The device of claim 33, further comprising one or more tissue anchorage features on the membrane frame, on one or more of the first and second struts, or on both the membrane frame and one or more of the first and second struts.

59. The device of claim 33, wherein the struts include atraumatic end portions.

60. The device of claim 33, wherein at least portions of the first and second struts or the membrane frame are coated with a coating that inhibits thrombus formation.

61. An implantable medical device for treating a portion of a body lumen wall, the device comprising:
a membrane frame;
a membrane material disposed on at least a portion of the membrane frame;
a first strut extending from the membrane frame; and
a second strut extending from the membrane frame, wherein the first and second struts are configured to press at least a portion of the membrane material into contact with the wall of the body lumen, and wherein the first and second struts each comprise an elongate member that is shaped as two arcuate segments and at least one wavy segment when the device is in a fully expanded configuration.

62. The device of claim 61, wherein the portion of the body lumen wall comprises a defect and the membrane material is configured to overlay the defect.

63. The device of claim 61, wherein the first and second struts each comprise elongate members that are unitary with an elongate member of the membrane frame.

64. The device of claim 61, wherein the first and second struts each comprise elongate members that are joined to an elongate member of the membrane frame.

65. The device of claim 61, wherein the first and second struts each comprise elongate members that are joined to an elongate member of the membrane frame by one or more of a weld, an adhesive, and a sleeve.

66. The device of claim 61, wherein the first and second struts extend from a perimeter of the membrane frame.

67. The device of claim 61, wherein the first and second struts have substantially identical shapes when the device is in a fully expanded configuration.

68. The device of claim 61, wherein the membrane material covers substantially the entire area defined by the membrane frame.

69. The device of claim 61, wherein the device assumes a low-profile configuration when the device is maintained in a constraining environment, and wherein the device expands from the low-profile configuration and assumes an expanded configuration when the device is liberated from the constraining environment.

70. The device of claim 61, wherein the membrane frame is configured to contact an arcuate portion of the body lumen wall of about 120 to 180 degrees.

71. The device of claim 61, wherein the membrane frame is configured to contact an arcuate portion of the body lumen wall of about 60 to 120 degrees.

72. The device of claim 61, wherein the membrane material is configured to inhibit tissue ingrowth and endothelialization into the membrane material.

73. The device of claim 61, wherein at least a portion of the membrane material is configured to be separated from the device and remain in a body when the device is removed from the body after treating the portion of the body lumen wall.

74. The device of claim 73, wherein at least a portion of the membrane material that is configured to be separated from the device is configured to promote tissue ingrowth or endothelialization into the membrane material.

75. The device of claim 74, wherein the portion of the membrane material that is configured to promote tissue ingrowth or endothelialization into the membrane material has an average porosity in the range of about 20 to 250 microns.

76. The device of claim 74, wherein the portion of the membrane material that is configured to promote tissue ingrowth or endothelialization into the membrane material has an average porosity in the range of about 100 to 200 microns.

77. The device of claim 74, wherein the portion of the membrane material that is configured to promote tissue ingrowth or endothelialization into the membrane material includes a plurality of openings that have an average diameter of about 0.25 to 2.0 millimeters.

78. The device of claim 74, wherein the portion of the membrane material that is configured to promote tissue ingrowth or endothelialization into the membrane material includes a coating of one or more growth factors.

79. The device of claim 61, further comprising an attachment feature configured for releasably coupling with a delivery device or a retrieval device.

80. The device of claim 61, wherein the membrane frame further comprises one or more elongate elements extending between two locations on a perimeter of the membrane frame.

81. The device of claim 80, wherein the one or more elongate elements extending between two locations on the perimeter of the membrane frame are non-linear.

82. The device of claim 61, further comprising one or more radiopaque markers on the membrane frame.

83. The device of claim 61, further comprising one or more radiopaque markers on one or more of the first and second struts.

84. The device of claim 61, wherein the portion of the body lumen wall comprises a defect and wherein the device is configured to prevent protrusion of the membrane material into the defect.

85. The device of claim 61, wherein the membrane frame has a length of about 0.5 to 15 centimeters.

86. The device of claim 61, further comprising one or more tissue anchorage features on the membrane frame, on one or more of the first and second struts, or on both the membrane frame and one or more of the first and second struts.

87. The device of claim 61, wherein the struts include atraumatic end portions.

88. The device of claim 61, wherein at least portions of the first and second struts or the membrane frame are coated with a coating that inhibits thrombus formation.

89. The device of claim 61, wherein the at least one wavy segment is disposed between the two arcuate segments.

90. A method of treating a defect in a body lumen wall, the method comprising:
inserting an implantable medical device into the body lumen using a transcatheter technique, the device comprising:
a membrane frame;
a membrane material disposed on at least a portion of the membrane frame;
a first strut extending from the membrane frame;
a second strut extending from the membrane frame, wherein the first and second struts are configured to exert opposing non-orthogonal forces to the membrane frame to thereby press at least a portion of the membrane material into contact with the wall of the body lumen; and
one or more radiopaque markers attached to the device;
wherein the membrane material is configured to overlay the defect.

91. The method of claim 90, further comprising visualizing the device during the inserting to confirm a positioning of the device as desired in relation to the defect.

92. The method of claim 91, further comprising detaching the device from a delivery device after confirming that the device has been positioned as desired in relation to the defect.

93. The method of claim 90, wherein the defect comprises an opening in the body lumen wall and the membrane material is configured to fully overlay the opening.

94. The method of claim 93, further comprising removing the device from the body lumen after the opening has been treated by the device.

95. The method of claim 94, further comprising reconfiguring the device in a low-profile configuration prior to removal of the device.

96. The method of claim 90, wherein the body lumen comprises an intestine and the defect comprises an opening in the wall of the intestine.

97. The method of claim 96, wherein the intestine is a colon.

98. The method of claim 90, wherein the body lumen is a blood vessel.

99. The method of claim 98, wherein the defect is an aneurysm in the blood vessel.

100. The method of claim 90, further comprising selecting the device based on a size of the defect.

101. The method of claim 90, further comprising delivering a pharmacological agent to treat the defect.

102. A method of treating a defect in a body lumen wall, the method comprising:
inserting a device into the body lumen using a transcatheter technique, the device comprising:
a membrane frame;
a membrane material disposed on at least a portion of the membrane frame;
a first strut extending from the membrane frame;
a second strut extending from the membrane frame, wherein the first and second struts are configured to press at least a portion of the membrane material into contact with the body lumen wall, and wherein the first and second struts each comprise an elongate member that extends from the membrane frame at two or more points and that forms a loop that is shaped as a generally elliptical segment when the device is in a fully expanded configuration; and
one or more radiopaque markers attached to the device;
wherein the membrane material is configured to overlay the defect.

103. The method of claim 102, further comprising visualizing the device during the inserting to confirm a positioning of the device as desired in relation to the defect.

104. The method of claim 103, further comprising detaching the device from a delivery device after confirming that the device has been positioned as desired in relation to the defect.

105. The method of claim 102, wherein the defect comprises an opening in the body lumen wall and the membrane material is configured to fully overlay the opening.

106. The method of claim 105, further comprising removing the device from the body lumen after the opening has been treated by the device.

107. The method of claim 106, further comprising reconfiguring the device in a low-profile configuration prior to removal of the device.

108. The method of claim 102, wherein the body lumen comprises an intestine and the defect comprises an opening in the wall of the intestine.

109. The method of claim 108, wherein the intestine is a colon.

110. The method of claim 102, wherein the body lumen is a blood vessel.

111. The method of claim 110, wherein the defect is an aneurysm in the blood vessel.

112. The method of claim 102, further comprising selecting the device based on a size of the defect.

113. The method of claim 102, further comprising delivering a pharmacological agent to treat the defect.

114. An implantable medical device for treating a portion of a body lumen wall, the device comprising:
a membrane frame;
a membrane material disposed on at least a portion of the membrane frame; and
two or more struts that each extend from the membrane frame and terminate at a free end, wherein the two or more struts are configured to exert non-orthogonal forces to the membrane frame to thereby press at least a portion of the membrane material into contact with the body lumen wall.

115. The device of claim 114, comprising four struts.

116. The device of claim 114, wherein the free ends are configured to be atraumatic ends.

117. The device of claim 114, wherein the two or more struts each comprise elongate members that are joined to an elongate member of the membrane frame.

118. The device of claim 114, wherein the two or more struts each comprise elongate members that are joined to an elongate member of the membrane frame by one or more of a weld, an adhesive, and a sleeve.

119. The device of claim 114, wherein the membrane frame further comprises one or more elongate elements extending between two locations on a perimeter of the membrane frame.

120. The device of claim 119, wherein the one or more elongate elements extending between two locations on the perimeter of the membrane frame are non-linear.

121. The device of claim 114, further comprising one or more radiopaque markers on the membrane frame.

122. The device of claim 114, further comprising one or more radiopaque markers on one or more of the two or more struts.
